(19) United States
(12) Patent Application Publication
(10) Pub. No.: US 2025/0152624 A1
(43) Pub. Date: May 15, 2025

(54) COMPOSITIONS AND METHODS OF TREATMENT FOR CANCERS

(71) Applicant: Washington University, St. Louis, MO (US)

(72) Inventors: Carl DeSelm, St. Louis, MO (US); Shelby Namen, St. Louis, MO (US)

(21) Appl. No.: 18/706,321

(22) PCT Filed: Nov. 2, 2022
(86) PCT No.: PCT/US2022/079187

§ 371 Date: Apr. 30, 2024

Related U.S. Application Data
(60) Provisional application No. 63/274,854, filed on Nov. 2, 2021.

(51) Int. Cl. A61K 35/15; A61K 40/19; A61K 40/31; A61K 40/42; A61P 35/00; A61P 37/04; C07K 16/28

(52) U.S. Cl. CPC A61K 35/15; A61K 40/19; A61K 40/31; A61K 40/4204; A61K 40/4211; A61K 40/4215; A61P 35/00; A61P 37/04; C07K 16/2827; C07K 16/2863; C07K 16/2866; C07K 16/2878; A61K 2239/13; A61K 2239/21; A61K 2239/22

(57) ABSTRACT

Among the various aspects of the present disclosure is the provision of compositions and methods of making modified chimeric antigen receptor dendritic cells (CAR-DCs) and methods of use thereof. CAR-DCs can be used for the treatment of tumors and cancers, particularly solid tumors (as well as liquid tumors, blood cancer, and metastatic cancer).

### SEQUENCE LISTING

This application contains a Sequence Listing that has been submitted in xml format via EFS-Web and is hereby incorporated by reference in its entirety. The xml copy is named 019995-PCT.xml and is about 23 KB in size.

### GOVERNMENTAL RIGHTS

This invention was made with government support under OD026427 awarded by the National Institutes of Health. The government has certain rights in the invention.

### FIELD OF THE TECHNOLOGY

Disclosed herein are compositions and methods of creating an adaptive immune response in a subject. In particular, the disclosure relates to dendritic cells genetically modified to express one or more chimeric antigen receptors (CARs) and methods of using the same for the treatment of cancer.

### BACKGROUND

Harnessing an organism's immune system to fight a disease such as cancer is a powerful approach. Recent work has identified two key immune checkpoint proteins displayed on the surface of T cells. These cell-surface receptors bind certain ligands displayed on other cells such as antigen-presenting cells (APCs) and recognize them as self, which leads to the attenuation of T cell activity and puts a brake on the immune response. Researchers have shown, during decades of work, that preventing these receptors on T cells from binding their ligands on APCs or tumor cells lifts the block and triggers an attack on the tumor cells.

For full activation of a T cell, this brake on negative immune modulation is necessary, but not sufficient. Another surface receptor, the T cell receptor (TCR), also needs to recognize and bind a ligand specifically derived from tumor cells and displayed on APCs. Evidence now exists that metastatic cancers are sometimes curable if a patient possesses antitumor T cells. Checkpoint inhibitors, which “release the breaks” on antitumor T cells, induce a complete response (CR) in up to 10-15% of metastatic melanoma and several other types of cancer, some of which are durable.

Chimeric antigen receptor (CAR) therapy has achieved great clinical success against hematological malignancies. It is based on synthetic receptors with both antigen recognition and signal transduction functions. The single-chain variable fragment (scFv) in a CAR retains its antigen recognition specificity from the variable regions of the heavy and light chains of the original monoclonal antibody. Meanwhile, signal transduction of the CAR construct largely depends on the signaling domains of the original immune receptors. CAR T cells exhibit a remarkable 80-100% CR in end-stage relapsed acute lymphoblastic leukemia (ALL) patients, but a 1% CR in solid tumors.

Understanding and overcoming the failures of each therapy may help bring durable remissions to the remainder of cancer patients. Reasons for failure are multifactorial, but checkpoint inhibitors are ineffective at baseline if patients do not first have an adaptive antitumor T cell response, which is more likely in tumors with higher mutational load. Solid tumors escape CAR T recognition if not all cells express the target antigen. Successfully creating an adaptive immune response in patients would overcome the failures of both types of immunotherapy. However, achieving this remains elusive.

Therefore, a need in the art exists for compositions and methods for specifically targeting various cancer or tumor cells, while creating an adaptive immune response by harnessing antitumor T cells.

### SUMMARY

In some aspects the current disclosure encompasses a Chimeric Antigen Receptor (CAR) comprising a intracellular signaling domain comprising an FMS-like tyrosine kinase 3 (Flt3) signaling domain or a functional fragment, variant or derivative thereof, and an extracellular domain no longer than 40 amino acids. In some aspects the extracellular domain is 5-20 amino acids in length, for example 5, 6, 7, 8, 9, 10, 11, 12, 13, 14, 15, 16, 17, 18, 19, or 20 amino acids in length. In some aspects the extracellular domain comprises a portion of a CD8 extracellular domain. In some aspects, the CAR comprises an extracellular domain comprising no more than 5, 6, 7, 8, 9, 10, 11, 12, 13, 14, 15, 16, 17, 18, 19, or 20 consecutive amino acids of a mouse or human CD8 extracellular domain. In some aspects, the extracellular domain comprises consecutive amino acids of SEQ ID NO: 8. In some aspects, the extracellular domain comprises a polypeptide sequence at least about 80% identical to SEQ ID NO: 7 or 18. In some aspects, the extracellular domain comprises consecutive amino acids of SEQ ID NOS. 14 or 15.

In some aspects, the intracellular domain of the CAR promotes survival and proliferation of dendritic cells. In some aspects, the intracellular signaling domain comprises a polypeptide sequence at least about 80% identical to SEQ ID NO: 5 or 6 or a functional fragment, variant or derivative thereof. In some aspects, the intracellular signaling domain comprises a human FMS-like tyrosine kinase 3 (Flt3) signaling domain or a functional fragment, variant or derivative thereof.

In some aspects, the CAR comprises an antigen binding domain. In some aspects, the antigen binding domain comprises a scFv. In some aspects, the antigen binding domain targets a tumor antigen. In some aspects, the tumor antigen is selected from a group consisting of EphA2, alphafetoprotein (AFP), carcinoembryonic antigen (CEA), CA-125, MUC-1 antibody, CD19, CD20, CD123, CD22, CD30, SlamF7, CD33, EGFRvIII, BCMA, GD2, CD38, PSMA, B7H3, EPCAM, IL-13Ra2, PSCA, Mesothelin, Her2, LewisY, LewisA, CIAX, epithelial tumor antigen (ETA), tyrosinase, melanoma-associated antigen (MAGE), abnormal products of ras or p53, or other proteins found to be more highly enriched on the surface of tumor cells than critical normal tissues. In some aspects, the tumor antigen is a human tumor antigen.

In some aspects, the CAR comprises a transmembrane domain. In some aspects, the transmembrane domain is at least about 80% identical to any one of SEQ ID. NOS: 11, 12, 13, 19 and 20.

In some aspects, the current disclosure also encompasses a polynucleotide comprising a nucleic acid sequence encoding a CAR described herein. In some aspects, the polynucleotide further comprises a promoter operably linked to the nucleic acid sequence encoding the CAR. In some aspects the promoter is a CD11c promoter or a variant or derivative thereof. In some aspects the promotor comprises a nucleic acid sequence at least about 60% identical to SEQ ID NO:1, SEQ ID NO:2, or SEQ ID NO:3. In some aspects, the polynucleotide can be a vector.

In some aspects, the current disclosure also encompasses a modified cell comprising a CAR disclosed herein and/or a polynucleotide as disclosed herein. In some aspects, the modified cell is an antigen presenting cell, for example a dendritic cell or a macrophage or a precursor thereof. In some aspects, the modified cell is a dendritic cell. In some aspects, the modified cell is selected from a conventional type 1 dendritic cell (cDC1) or a progenitor cell thereof. In some aspects the modified cell is not a monocyte derived dendritic cell. In some aspects, the progenitor cell is selected from a peripheral blood mononuclear cell (PBMC), a monocyte and dendritic cell progenitor (MDP), a common myeloid progenitor (CMP), a lymphoid-primed multipotent progenitor (LMPP) or a common dendritic cell progenitor (CDP), or a stem cell, such as a CD34+ hematopoietic stem cell. In some aspects the modified cell is capable of cross presenting a tumor antigen, eliciting an adaptive antitumor immune response, or activating antitumor T cells. In some aspects, the modified cell is capable of selectively engulfing tumor cells, cross-presenting a tumor antigen, or activating T-cells to respond to the tumor antigen. In some aspects, the modified cell is capable of eliminating antigen positive (Ag+) tumors targeted by the CARs, and indirectly eliminating CAR-Ag-solid tumor cells (not recognized by the CAR), through epitope spreading.

In some aspects, the current disclosure also encompasses a pharmaceutical composition comprising a CAR as disclosed herein or modified cell or a population of modified cells as disclosed herein, or a polynucleotide as disclosed herein and a pharmaceutically acceptable excipient. In some aspects the pharmaceutical composition may comprise a CAR as disclosed herein incorporated into a nanoparticle.

In some additional aspects, the current disclosure encompasses a method of treating cancer in a subject in need thereof, comprising administering to the subject a therapeutically effective amount of a pharmaceutical composition as disclosed herein. In some aspects, the subject has a cancer, wherein the cancer is a malignant tumor, solid tumor, or liquid tumor. In some aspects, the subject is a human.

In some aspects, the pharmaceutical composition or the cells as disclosed herein can be administered through one or more of a parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal route. In some aspects, the composition is administered daily, weekly, bi-monthly, monthly less frequently during a treatment period. In some aspects, additional treatments selected from chemotherapy, radiation therapy, or immunotherapy may be combined with the method. In some aspects, administering the pharmaceutical composition induces a tumor reducing immune response. In some aspects, the administering of the pharmaceutical composition induces phagocytosis of cancer cells in the subject. In some aspects of the disclosed method, the modified cell of the composition cross-primes an anti-tumor T-cell response. In some aspects, the modified cell of the composition creates a tumor-eliminating immune response. In some aspects, the modified cell of the composition directly targets antigen positive (Ag+) tumor cells for elimination; or indirectly targets CAR-antigen negative (Ag−) tumor cells for elimination through cross-presentation and epitope spreading.

In some aspects the current disclosure also encompasses use of the modified cells disclosed herein for treatment of cancer. In some aspects, the current disclosure also encompasses use of the modified cells as disclosed herein in the manufacture of a medicament for the treatment of cancer.

In some aspects, the current disclosure encompasses chimeric antigen receptor (CAR), comprising from N-terminus to C-terminus: a signal peptide, an antigen binding domain, an extracellular domain no longer than 40 amino acids, a transmembrane domain; and an intracellular signaling domain comprising an FMS-like tyrosine kinase 3 (Flt3) signaling domain or a functional fragment, variant or derivative thereof. In some aspects, the signal peptide comprises a polypeptide sequence at least about 80% identical to SEQ ID NO: 9 or 10. In some aspects, the antigen binding domain targets a tumor antigen selected from the group consisting of BCMA, EphA2, B7H3, CD19 and EGFRvIII. In some aspects, the antigen binding domain comprises a polypeptide sequence at least about 80% identical to SEQ ID NO: 4. In some aspects, the extracellular domain comprises a polypeptide sequence at least about 80% identical to SEQ ID NO: 7 or 18. In some aspects, the extracellular domain comprises a polypeptide sequence comprising 5-20 consecutive amino acid residues of any one of SEQ ID NO: 7, 8, 14, 15 or 18. In some aspects, the intracellular domain comprises a polypeptide sequence at least about 80% identical to SEQ ID NO: 5, or 6 or a fragment, variant or derivative thereof. In some aspects, the CAR further comprising a linker. In some aspects, the linker comprises a polypeptide sequence at least about 80% identical to SEQ ID NO: 16. In some aspects, the CAR consists of the signal peptide, antigen binding domain, extracellular domain, transmembrane domain, and intracellular signaling domain.

In some aspects, the current disclosure also encompasses kits comprising the compositions disclosed herein and instructions.

In an exemplary aspect, the current disclosure encompasses a human CAR comprising an extracellular domain comprising an amino acid sequence no longer than 13 amino acids, a tumor antigen binding domain (for example, a tumor antigen binding domain that selectively binds to BCMA, CD19, EGFRvIII, EphA2 or B7H3) and a transmembrane domain comprising an amino acid sequence from human CD8 or CD8b transmembrane domain, and an intracellular domain comprising an amino acid sequence from human Flt3.

In some aspects, CAR comprises an (a) extracellular domain comprising the amino acid sequence set forth in SEQ ID NO: 7, 8 or 18, (b) a tumor antigen-binding domain; (c) a transmembrane domain comprising the amino acid sequence set forth in SEQ ID NO: 12, 13 or 20 and (c) an intracellular domain comprising the amino acid sequence set forth in SEQ ID NO: 6 or a fragment, variant, or derivative thereof.

Other objects and features will be in part apparent and in part pointed out hereinafter.

### BRIEF DESCRIPTION OF THE FIGURES

Those of skill in the art will understand that the drawings, described below, are for illustrative purposes only. The drawings are not intended to limit the scope of the present teachings in any way.

FIG. 1A shows that CAR T-cell therapy is unable to efficiently target solid tumors.

FIG. 1B provides an alternate approach where CAR encoding antigen presenting cells can cross-present endogenous tumor antigen in a manner that cross-primes tumor antigen-reactive T-cells.

FIG. 2A shows a schematic of the construct comprising a EphA2 CAR and RFP. EphA2 is expressed on a range of tumors. FACS data shows that EphA2 is naturally expressed on the 1956 sarcoma tumor used in these experiments.

FIG. 2B is a schematic of the EphA2 CAR construct followed by a p2A cleavage sequence and RFP. Potential regions for optimization are indicated.

FIG. 3A shows that the EphA2/FcGR CAR, which is an ITAM-signaling based CAR utilizing the common gamma chain of the Fc receptor, can be successfully transduced into bone marrow macrophages.

FIG. 3B is a bright field image showing bone marrow macrophages containing an EphA2/FcGR CAR (FcGR CAR MACS) in the presence of tumor cells.

FIG. 3C is a fluorescence image showing that FcGR CAR MACS can successfully target tumor cells.

FIG. 3D is a graph showing in vivo data that FcGR CAR MACS can cause a statistically significant shrinkage of tumor size in syngeneic mice model for single sarcoma (p<0.05).

FIG. 3E is a graph showing in vivo data that FcGR CAR MACS can reduce tumor size or burden on the treated side in dual flank sarcoma mouse models.

FIG. 3F is a graph showing in vivo data that FcGR CAR MACS do not reduce tumor size or burden on the untreated side in dual flank sarcoma mouse models compared with control macrophages that have been transduced with a vector containing the red fluorescent protein with no CAR.

FIG. 3G is a graph showing in vivo data that FcGR CAR MACS are not able to reduce tumor size on the treated side in dual flank sarcoma mouse models on rechallenge, showing that no T-cell memory is established by the treatment with FcGR CAR MACS.

FIG. 3H shows a schematic of the construct use to test if FcGR signaling negatively impacts cDC1 differentiation. The figure further shows FACS based dendritic cell differentiation data for control and CAR transduced cells. Data shows the FcGR CAR transduced cells exhibit inhibited differentiation of conventional type 2 dendritic cells (cDC2) but had impaired ability to differentiate into conventional type 1 dendritic cells (cDC1).

FIG. 4A shows a schematic of new CAR sequences designed to comprise an extracellular domain (EC) comprising a FLAG tag and 5-35 residues of the CD8 extracellular domain, followed by a transmembrane domain and a Flt3 intracellular domain (IC).

FIG. 4B shows in vivo data showing tumor induced CAR cDC1 differentiation and survival from bone marrow mononuclear cells comprising the new CAR sequences with the short extracellular domain.

FIG. 4C shows a bar graph showing percentage of cDC1 differentiation from MACS precursor cells, isolated from the bone marrow, expressing CARs with either a TCR-based ITAM domain, FcR or Flt3 IC. Statistically significant enhancement of differentiation is shown with Flt3 IC compared to precursors with FcR CAR or untransduced MACS.

FIG. 4D are brightfield and fluorescent images showing the effect of Flt3 IC CARs on dendritic cell survival and expansion in the presence of tumor cells with or without exogenous Flt3L.

FIG. 5A shows a schematic of the CAR constructs comprising long and short IC versions of the CD8 intracellular domains. HoxB8 pluripotent DC precursor cells transduced with either of the two constructs showed similar CAR expression as evident from FACS analysis.

FIG. 5B is a bar graph showing the importance of EC domain length on survival of DC precursors when grown in the presence of tumor without any exogenous Flt3L or other cytokines. As expected, all untransduced cells died. The long EC CAR exhibit significantly improved survival when grown on tumor. However, the short EC CAR performed significantly better in survival assays compared to the long EC CAR.

FIG. 5C is a schematic and bar graph showing that increased survival of tumors mediated by a short EC CAR was due to the EC CAR and not due to the EC domain (ECD) alone. A short CAR was compared to an identical version that lacked the intracellular signaling domain in tumor co-culture. The short EC CAR lacking a signaling domain had no better survival than control untransduced cells, while short EC CAR with the Flt3 signaling domain again had substantially improved survival.

FIG. 5D is a bar graph showing the importance of EC domain length on survival of DC precursors in the presence or absence of Flt3L, by quantifying the change in CAR-high expressing cells in the presence or absence of tumor and/or Flt3L. In the presence of exogenous Flt3L, all DC precursors survive and proliferate similarly, and the number of high CAR expressing cells does not substantially change. However, when tumor cells replace exogenous Flt3L, the DC precursors that receive an effective survival signal through the Flt3 CAR recognizing the tumor have a notable survival advantage. The long EC CAR exhibited significantly improved survival when grown on tumors without Flt3L. However, the short EC CAR performed significantly better in survival assays compared to the long EC CAR when grown on tumor in the absence of Flt3L.

FIG. 5E shows FACS data showing CAR mediated tumor antigen uptake. To test whether this DC CAR induces any tumor uptake upon binding, we cocultured CAR HoxB8 DC cells (differentiated with Flt3L on OP9-DL1 cells as described by Kirkling et al, Cell Reports 2018 PMID 29925006) with tumor expressing zsGreen for 48 hours.

FIG. 5F is a bar graph showing that the cells expressing short EC CAR exhibit higher tumor phagocytosis compared to cells expressing long EC CAR. The absolute number of cells taking up tumor (after plating equivalent numbers) was higher with short EC CAR.

FIG. 5G is a bar graph showing that the short EC CAR dendritic cells promote substantial amount of tumor uptake, while the long EC CAR cDC1 results in less tumor uptake, and the non-signaling short CAR and untransduced cells both exhibit very limited tumor uptake (2-4%).

FIG. 6A provides FACS data showing proliferation of CAR cDC1 when the CAR is expressed from a MulV (control), BATF3, CD11c v1 or CD11c v2 promoter.

FIG. 6B is a bar graph showing % cDC1 cells in the DC population when CAR is expressed from MulV (control), BATF3, CD11c v1 or CD11c v2 promoter.

FIG. 7A provides a graph showing the tumor burden as estimated from tumor size for tumors directly injected with short EC CAR DCs. Sarcoma was orthotopically injected into the bilateral flank of syngeneic mice, and once established, one of the two tumors in each mouse was injected with three million control or Flt3 CAR cDC1s with a short extracellular domain. FIG. 7B provides a graph showing the tumor burden as estimated from tumor size for injected (treated) tumors.

FIG. 7B provides a graph showing the tumor burden as estimated from tumor size for untreated tumors from the same mice described in FIG. 7A.

FIG. 7C provides a graph showing the tumor burden as estimated from tumor size. Flt3 CAR DC-treated mice exhibiting complete tumor response were re-injected with tumor, and tumor burden measured.

FIG. 8A shows a schematic of the CD19-CAR and EGFRvIII-CAR comprising the Flt3 intracellular signaling domain and the short of long versions of the extracellular domains.

FIG. 8B shows a schematic of the cross priming assay combining ovalbumin loaded target cells that express either human CD19 or EGFRvIII and do not express MHCI with CAR⁺ DCs, cell trace violet (CTV) stained naïve OTI (TCR transgenic CD8 T cells that recognize MHCI:SIINFEKL (SEQ ID NO:17) T cells, and poly-IC (TLR3 agonist). The cells were subjected to flow cytometry after 72 hours to look for CTV dilution showing T cell proliferation due to cross-priming.

FIG. 9A shows a schematic of the cross-priming assay with anti-CD19 short or long Flt3-based CAR DCs or untransfected (UNTF) DCs cultured with irradiated (60 Gy) ovalbumin loaded mRuby⁺hCD19⁺ human embryonic kidney (HEK293) cells and naïve CTV+ OTI T cells with Poly-IC.

FIG. 9B shows representative flow cytometry plots of proliferated OTI cells 72 hours after co-culture gated on CD45.1⁺mRuby⁻CD8a⁺TCRVa2⁺.

FIG. 9C shows quantitative data showing proliferation of OVA-specific OTI CD8⁺ T cells(cell/ul) in response to various numbers of OVA-loaded HEK293 cells presented by UNTF (black), short CD19 CAR (blue), or long CD19 CAR (red).

FIG. 9D shows bar graphs providing percentages and cells per microliter of CTV diluted OTI T cells in a range of assays with varying numbers of target cells, while keeping the CAR DC and T cell numbers constant. n=3.

FIG. 10A shows a schematic of the cross-priming assay with anti-EGFRvIII short or long Flt3-based CAR DCs or untransfected (UNTF) DCs cultured with irradiated (60 Gy) ovalbumin loaded zsGreen⁺EGFRvIII⁺B2M⁻ mouse 1956 tumor cells and naïve CTV+OTI T cells with Poly-IC.

FIG. 10B shows representative flow cytometry plots of proliferated OTI cells 72 hours after co-culture gated on CD45.1⁺zsGreen⁻CD8a⁺TCRVa2⁺.

FIG. 10C shows bar graphs providing proliferation percentages and cell counts of OVA-specific OTI CD8⁺ T cells in both percent and cell per microliter in response to different numbers of OVA-loaded 1956 cells presented by UNTF (grey), short EGFRvIII CAR (light blue), or long EGFRvIII CAR (pink). N=3.

FIG. 11A shows a schematic of the cross-priming assay with anti-EGFRvIII short or long Flt3-based CAR DCs or untransfected (UNTF) DCs cultured with non-irradiated ovalbumin loaded zsGreen⁺EGFRvIII⁺B2M⁻ mouse 1956 tumor cells and naïve CTV+OTI T cells with Poly-IC.

FIG. 11B shows representative flow cytometry plots of proliferated OTI cells 72 hours after coculture gated on CD45.1⁺zsGreen⁻CD8a⁺TCRVa2⁺.

FIG. 11C shows bar graphs providing proliferation data for OVA-specific OTI CD8⁺ T cells in both percent and cell per microliter in response to different numbers of OVA-loaded 1956 cells presented by UNTF (grey), short EGFRvIII CAR (light blue), or long EGFRvIII CAR (pink). N=3.

FIG. 12A shows a schematic of in vivo mice survival experiments. C57BL/6 mice were injected bilaterally in the flank with 1×10⁶ human CD19+ mouse 1956 tumor cells and one day later 1.7×10⁶ CAR or control transfected cDC1s that had been differentiated in vitro with Flt3L containing media from primary bone marrow-derived cells into conventional DCs (bulk unsorted; containing 2.7×10⁶ cDC2s). Survival of the mice and both tumor sizes were measured until the tumors reached a diameter of two centimeters at which time the mice were euthanized.

FIG. 12B shows survival data for mice injected bilaterally with 1×10⁶ human CD19+ mouse 1956 tumor cells and one day later injected with 1.6×10⁶ unsorted short CD19 CAR cDC1s (blue), unsorted long CD19 CAR cDC1s (red), 1.6×10⁶ untransfected (UNTF) cDC1s (green), or nothing (untreated) (black). short CD19 CAR cDCs, n=5; unsorted long CD19 CAR cDCs, n=2; UNTF cDCs, n=5; untreated, n=3.

### DETAILED DESCRIPTION

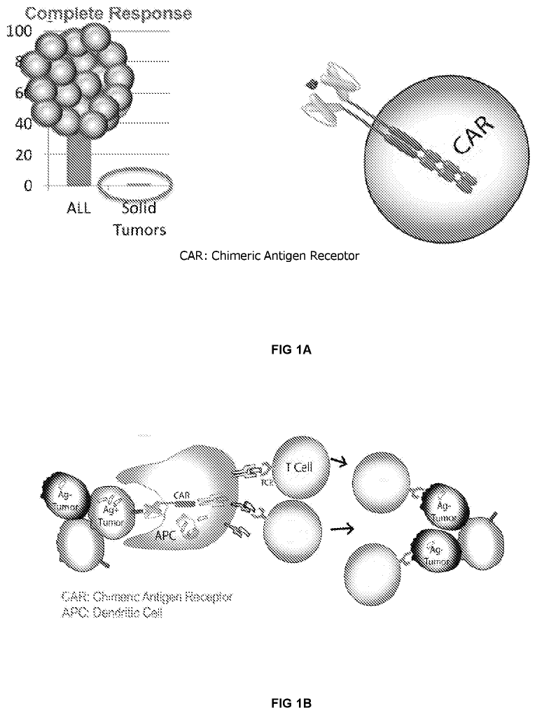

The present disclosure is based, at least in part, on the discovery that dendritic cells that have been genetically modified to express a chimeric antigen receptor (CAR) are capable of targeting tumor cells for phagocytosis and cytotoxicity through T-cell cross-priming. As shown herein, CAR dendritic cells (CAR-DCs) can be used to treat various cancers and malignancies, including solid tumors. Previously described CAR macrophages (CAR-Ms) have not successfully cross-primed T-cells after phagocytosing or pinocytosing tumor cells, and have not been successful in eliminating solid tumors in vivo (see FIGS. 1A and 1). The present disclosure describes a method of generating functional CAR-DCs, which selectively engulf tumor cells and cross-present endogenous tumor antigen in a manner that cross-primes tumor antigen-reactive T-cells. The disclosure demonstrates that CAR-DCs derived from a Flt3-based CAR are able to successfully generate cDC1 cells, whereas traditional Fc receptor based CARs introduced into myeloid precursor cells do not form cDC1s, but rather form macrophages, even when they are grown in the presence of the DC-differentiating cytokine Flt3L. The inability of non-Flt3-based CARs to successfully generate DCs appears to be due to basal signaling from the CAR that impairs proper differentiation to the cDC1 phenotype. The present disclosure thus provides compositions and methods for creating an adaptive immune response using CAR DCs. The adaptive immune response generated is useful to target and kill both CAR-Ag+ and Ag-tumor or cancer cell, as well as create an immune memory which prevents tumor or cancer cell recurrence.

A composition of the disclosure may optionally comprise one or more additional drugs or therapeutically active agents in addition to the CAR-DCs. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, salts (substances of the present invention may themselves be provided in the form of a pharmaceutically acceptable salt), buffers, coating agents, or antioxidants.

Other aspects and iterations of the invention are described more thoroughly below.

### I. Compositions

For the first time, as described herein, is a CAR construct that allows for the generation of functional CAR-containing cells. In one example, the CAR-containing cells are CAR-DCs. The CAR-containing cells may selectively engulf tumor cells and cross-present endogenous tumor antigens in a manner that cross-primes endogenous tumor antigen-reactive T-cells to eliminate remaining tumor, in vitro and in vivo.

Previous work has described CAR-macrophages. Macrophages, like dendritic cells, can phagocytose material and can present antigens. However, in vivo, macrophages are unable to effectively cross-present tumor antigens, and are unable to create a tumor-eliminating immune response through T-cell cross-priming. In vivo, DCs, and particularly the subset of DCs known as cDC1s, are the only cells capable of tumor antigen cross-presentation and T-cell cross-priming in vivo; without cDC1s in vivo, an adaptive antitumor response is not achievable, an anti-tumor immune response cannot be mounted, and tumor cannot be eliminated by the immune system in vivo. Thus, conceptually, CAR-macrophages can achieve the goal of direct tumor phagocytosis or possibly direct cellular cytotoxicity. However, they cannot achieve the goal of antigen cross-presentation and cannot create an effective adaptive anti-tumor T cell response.

CAR-macrophages have been created by fusing the intracellular domain of various macrophage receptors that induce phagocytosis, such as Fc receptors, toll-like receptors, or other macrophage or T-cell based receptors, with a tumor-recognizing scFv extracellular domain. No CARs to date have successfully been created that endow cells with DC capacity; that is, the ability to cross-prime an effective anti-tumor T-cell response, particularly in vivo.

### (a) CAR-Containing Cells

The present disclosure provides chimeric antigen receptor-bearing cells, which may be dendritic cells (CAR-DCs), pharmaceutical compositions comprising them, and methods of immunotherapy for the treatment of cancers or tumors. A CAR-DC is a dendritic cell which expresses a chimeric antigen receptor (CAR). As described herein, dendritic cells, or precursors or progenitors thereof, can be modified to form CAR dendritic cells (CAR-DCs). A CAR is a recombinant fusion protein comprising: 1) an extracellular ligand-binding domain, i.e., an antigen-recognition or antigen binding domain, 2) a transmembrane domain, and 3) a signaling transducing domain.

CAR-DCs comprising a Flt3-based CAR construct are able to functionally phagocytose tumor cells in a CAR-dependent manner and cross-prime anti-tumor T-cells by tumor uptake and antigen cross-presentation, whereas CAR-macrophages cannot. Thus, the term includes DCs that initiate an immune response and/or present an antigen to T lymphocytes and/or provide T-cells with any other activation signal required for stimulation of an adaptive immune response.

As described herein, CAR-DCs can be generated by exposing isolated dendritic cell progenitors, such as stem cells (pluripotent, multipotent, hematopoietic, or other stem cells), multipotent progenitors, common myeloid progenitors (CMP), myeloid dendritic cell progenitors (MDP), common dendritic cell progenitor (CDP), bone marrow mononuclear cells, peripheral blood mononuclear cells (PBMC), or splenocytes, to a DC proliferative stimulus such as Flt3L. The cell can then be transduced with the CAR of interest and further exposed to the DC differentiating factor Flt3L for an amount of time sufficient to generate dendritic cell-like cells (DC-like cells) prior to treatment. For example, the cell can be exposed to Flt3L for about 2 to 15 days to promote differentiation.

The present disclosure provides for modified dendritic cells (DCs) and modified precursors and modified progenitors of DCs. DCs are immune cells that are capable of antigen cross-presentation and are critical in initiating an adaptive immune response, particularly to tumor. Numerous studies demonstrate that DCs are limited in the tumor microenvironment and even in cancer patients in general. Further, even if DCs are present, they can induce tolerance or rejection of an antigen, or have no effect at all as they generally have no strong signal instructing them a tumor cell is foreign or threatening and in need of being eliminated.

Additional description of the modified DCs, modified precursors, and modified progenitors of DCs may be found in PCT Application No. PCT/US2020/065378, the content of which is incorporated herein in its entirety.

A dendritic cell can be a subset of dendritic cells. As an example, a subset of DCs can be, for example, plasmacytoid DC (pDC), myeloid/conventional/classical DC1 (cDC1), myeloid/conventional/classical DC2 (cDC2), or monocyte-derived DC (moDC).

A progenitor cell can be any cell that is capable of differentiating into a DC. For example, a DC progenitor can be a stem cell (pluripotent, multipotent, hematopoietic, or other stem cell), multipotent progenitor, common myeloid progenitor (CMP), myeloid and dendritic cell progenitor (MDP), a lymphoid-primed multipotent progenitor (LMPP), common dendritic cell progenitor (CDP), bone marrow monocytes, a peripheral blood mononuclear cell (PBMC), or splenocytes.

A precursor of DCs can be a progenitor cell, as described above, or any cell that can be induced or reprogramed to differentiate into DCs, such as fibroblasts. For example, precursors to DCs can be stem cells, monocytes, myeloid precursor cells, myeloid-derived precursor cells, peripheral blood mononuclear cells (PBMCs), or bone marrow monocytes (BMM).

CAR-DCs can be autologous, meaning that they are engineered from a subject's own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

The present disclosure provides for modified conventional type I dendritic cells (cDC1s), which can be generated by differentiating CAR-DCs. As described herein, in vivo, DCs, and particularly the subset of DCs known as cDC1s, are the only immune cells capable of effective tumor antigen cross-priming. Antigen cross-priming refers to the stimulation of antigen-specific naïve cytotoxic CD8 T-cells into activated cytotoxic CD8 T-cells by antigen presenting cells that have acquired and cross-presented extracellular antigen, in this case acquired from tumor. Antigen cross-presentation refers to the ability of a cell to present internalized antigens on type I major histocompatibility complex molecules (MHC I). Antigen cross-presentation and cross-priming are known to be necessary for an efficient adaptive immune response against tumor cells.

Without cDC1s, an adaptive antitumor response is not achievable, an anti-tumor immune response cannot be mounted, and tumor cannot be eliminated by the immune system in vivo, see, e.g. the below Examples.

As described herein, transducing a dendritic cell or precursor thereof with a Flt3-based CAR is uniquely able to produce true, programmable and functional cDC1s, which has not been before demonstrated. Traditional Fc receptor-based CARs introduced into myeloid precursor cells do not form cDC1s, but rather form macrophages or cDC2s, even though they are grown in the cDC1-differentiating cytokine Flt3L. The inability of non-Flt3-based CARs to successfully generate DCs appears to be due to basal signaling from the CAR that impairs proper differentiation to the cDC1 phenotype. Thus, Flt3-based CARs can be called “CAR-DCs,” which are to be distinguished from other CARs (based on the Fc receptor or other inflammatory or macrophage receptor domains), which when expressed in progenitor cells produce CAR-macrophages (CAR-Ms) that possess significantly inferior ability to cross-prime T-cells. Unlike CAR-Ms or cDC1s, CAR-DCs have superior ability to selectively engulf tumor cells, cross-present tumor antigen, and activate T-cells to respond to the tumor antigen.

As described herein, cDC1s can be identified based on flow cytometry for specific surface protein expression signatures and confirmed by their functional capacity to cross-prime T-cells against engulfed cell-associated antigen. For example, the cDC surface expression profile can be lineage-negative B220⁻, CD11c⁺, and MHC-II⁺, and cDC1 and cDC2s can be further differentiated by CD24 and Sirpa expression.

### (b) CAR Constructs

CAR designs are generally tailored to each cell type. The present disclosure is drawn to dendritic cells or precursors thereof but could be useful in other immune cell types and precursors thereof, like bone marrow macrophages. Disclosed herein are dendritic cells engineered to express chimeric antigen receptors (CARs).

CARs are designed in a modular fashion that comprise a signal peptide, an extracellular target-binding domain (e.g., antigen-binding domain, tumor binding domain, or a ligand-binding domain), an extracellular domain, a transmembrane domain that anchors the CAR to the cell membrane, and one or more intracellular domains that transmit activation signals. In some aspects, the CAR may also comprise one or more linker sequences. In one embodiment, the CAR has the following structure: signal peptide—antigen binding domain—extracellular domain—transmembrane domain—intracellular domain and one or more linker sequences.

A signal peptide directs the transport of a secreted or transmembrane protein to the cell membrane and/or cell surface to allow for correct localization of the polypeptide. Particularly, the signal peptide of the present disclosure directs the appended polypeptide, i.e., the CAR, to the cell membrane wherein the antigen binding domain of the appended polypeptide is displayed on the cell surface, the transmembrane domain of the appended polypeptide spans the cell membrane, and the signaling transducing domain of the appended polypeptide is in the cytoplasmic portion of the cell. Any suitable signal peptide can be used in the CAR sequences of the current disclosure. In one embodiment, the signal peptide is the signal peptide from human CD8α A functional fragment is defined as a fragment of at least 10 amino acids of the CD8α signal peptide that directs the appended polypeptide to the cell membrane and/or cell surface. In some aspects, the signal peptide of the CAR may comprise an amino acid sequence at least about 80% identical to SEQ ID. NO: 9 or 10. After a cell processes the CAR, the signal peptide may be removed from the CAR.

As described herein, the CAR can comprise an antigen binding domain or tumor binding domain (also referred to as an extracellular ligand-binding domain or a ligand binding domain). The antigen binding domain can comprise any domain that binds to an antigen expressed by the targeted cell type (e.g., an antigen expressed by a tumor cell) or a fragment thereof (see e.g., Saar Gill et al. U.S. application Ser. No. 15/747,555 incorporated herein by reference in its entirety). For example, the antigen binding domain can be an antibody (from human, mouse, or other animal), a humanized antibody, a monoclonal antibody, a polyclonal antibody, a synthetic antibody, a camelid antibody, a native receptor or ligand, or a fragment thereof. For example, the antigen binding domain can be a single-chain variable fragment (scFv) of an antibody. The antigen binding domain can be directed to various tumor associated proteins, which may include EphA2, alphafetoprotein (AFP), carcinoembryonic antigen (CEA), CA-125, MUC-1 antibody, CD19, CD20, CD123, CD22, CD30, SlamF7, CD33, EGFRvIII, BCMA, GD2, CD38, PSMA, B7H3, EPCAM, IL-13Ra2, PSCA, Mesothelin, Her2, LewisY, LewisA, CIAX, epithelial tumor antigen (ETA), tyrosinase, melanoma-associated antigen (MAGE), abnormal products of ras or p53, or other proteins found to be more highly enriched on the surface of tumor cells than critical normal tissues. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A and ERBB. In particular, the tumor antigen may be CD19, B7H3, EGFRvIII or EphA2.

The antigen binding domain may be a scFv. Targeting antibody fragments or scFvs, as described herein, can be against any disease-associated antigen or tumor-associated antigen (TAA). A TAA can be any antigen known in the art to be associated with tumors.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

The antigen-binding capability of the CAR is defined by the extracellular scFv. The format of a scFv is generally two variable domains linked by a flexible peptide sequence, either in the orientation V_{H}-linker-V_{L} or V_{L}-linker-V_{H}. Suitable linker sequences are described elsewhere in this disclosure. The orientation of the variable domains within the scFv, depending on the structure of the scFv, may contribute to whether a CAR will be expressed on the dendritic cell surface or whether the CAR-DCs target the antigen and signal. In addition, the length and/or composition of the variable domain linker can contribute to the stability or affinity of the scFv. In some aspects, the scFv may comprise an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or greater identity to an amino acid sequence of the antigen binding domain targeting CD19, EGFRvIII, or EphA2 In some aspects, the scFv may have the amino acid sequence set forth in SEQ ID NO: 4, or a functional variant, derivative, or fragment thereof. In some aspects, the scFv may have the amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence set forth in SEQ ID NO: 4, or a functional variant, derivative, or fragment thereof.

The scFv, a critical component of a CAR molecule, can be carefully designed and manipulated to influence specificity and differential targeting of tumors versus normal tissues.

In some exemplary embodiments, the CAR has the following structure: signal peptide—antigen binding domain directed to a tumor antigen selected from BCMA, EphA2, B7H3, CD19 and EGFRvIII—extracellular domain—transmembrane domain—intracellular domain.

Typically, the antigen binding domain is linked to the signaling transducing domain (i.e., intracellular domain) of the chimeric antigen receptor (CAR) by a transmembrane domain (Tm). The transmembrane domain traverses the cell membrane, anchors the CAR to the DC surface, and connects the antigen binding domain to the signaling transducing domain, impacting the expression of the CAR on the DC surface. The distinguishing feature of the transmembrane domain in the present disclosure is the ability to be expressed at the surface of a DC to direct an immune cell response against a pre-defined target cell. The transmembrane domain can be derived from natural or synthetic sources. Alternatively, the transmembrane domain of the present disclosure may be derived from any membrane-bound or transmembrane protein.

Non-limiting examples of transmembrane polypeptides of the present disclosure include CD8 alpha (CD8a) or beta (CD8b), alpha, beta or zeta chain of the T-cell receptor, CD28, CD3 epsilon, CD45, CD4, CDS, CD9, CD16, CD22, CD33, CD37, CD64, CDSO, CD86, CD134, CD137 and CD154. Alternatively, the transmembrane domain can be synthetic and comprise predominantly hydrophobic amino acid residues (e.g., leucine and valine). In some aspects, the transmembrane domain is at least about 80% identical to any one of SEQ ID NOS: 11, 12, 13, 19 and 20, or a functional fragment, variant, or derivative thereof.

In some exemplary embodiments, the CAR has the following structure: signal peptide—antigen binding domain—extracellular domain—transmembrane domain selected from SEQ ID NO: 11, 12, 13, 19 or 20 functional fragment, variant, or derivative thereof—intracellular domain.

The CAR may comprise an extracellular domain. The “extracellular domain” or “spacer” may link the transmembrane domain and the antigen-binding domain (i.e., ligand-binding domain). The extracellular domain is in the extracellular structural region of the CAR that separates the binding units from the transmembrane domain. The extracellular domain can be any moiety capable of ensuring proximity of a CAR-containing cell to its target (e.g., CD8-based hinge). In some aspects, the extracellular domain provides flexibility to access the targeted antigen. The optimal length of the extracellular domain may depend on the position of the targeted epitope. Long spacers can provide extra flexibility to the CAR and allow for better access to membrane-proximal epitopes or complex glycosylated antigens. CARs bearing short extracellular domains can be more effective at binding membrane-distal epitopes. The length of the extracellular domain may provide adequate intercellular distance for immunological synapse formation. As such, the extracellular domain may be optimized for an individual epitope. With the exception of CARs based on the entire extracellular moiety of a receptor, the majority of CAR (such as CAR T) cells are designed with immunoglobulin (Ig)-like domain spacers or CD8 spacers, but any protein sequence that proves a space between the transmembrane domain and target-binding domain may function as an effective extracellular domain.

In some aspects, the current disclosure encompasses a CAR comprising an extracellular domain no longer than 40 amino acids. In some aspects the extracellular domain is 5-30 or 5-20 amino acids in length, for example 5, 6, 7, 8, 9, 10, 11, 12, 13, 14, 15, 16, 17, 18, 19, 20, 21, 22, 23, 24, 25, 26, 27, 28, 29, or 30 amino acids in length, or a range of the foregoing. An extracellular domain of these lengths in combination with a Flt3 intracellular domain disclosed herein may result in improved activity of the CAR. In some aspects the extracellular domain comprises a portion of a CD8 extracellular domain. In some aspects, the CAR comprises consecutive amino acids of a mouse or human CD8 extracellular domain. In some aspects the CD8 extracellular domain comprises a polypeptide sequence at least about 80% identical to SEQ ID NO: 7 or 8. The extracellular domain may have the sequence set forth in SEQ ID NO: 7. In another aspect, the extracellular domain comprises a polypeptide sequence at least 80% identical to SEQ ID NO: 18. In another, the extracellular domain has the sequence set forth in SEQ ID NO: 18. In some aspects, the extracellular domain comprises consecutive amino acids of SEQ ID NO: 14 or 15.

In some aspects, short extracellular domains as disclosed herein can supply stability for efficient CAR expression and activity. The extracellular domain (also in combination with the transmembrane domain), also ensures proper proximity to target.

In some aspects, the extracellular domain may be operably linked to the transmembrane domain.

Optionally, an extracellular signaling domain may be incorporated into the CAR construct to propagate signaling. The extracellular signaling domain may be cloned into the extracellular region, but can be chosen based on the target.

In some embodiments, the CAR has the following structure: signal peptide—antigen binding domain (with a linker sequence connecting the variable regions)—extracellular domain≤13 amino acids in length-transmembrane domain—intracellular domain.

A chimeric antigen receptor (CAR) of the present disclosure comprises a signal transducing domain or intracellular signaling domain that is responsible for intracellular signaling following the binding of the extracellular antigen binding domain to the target resulting in the activation of the immune cell and immune response. In other words, the signal transducing domain is responsible for the activation of at least one of the normal effector functions of the immune cell in which the CAR is expressed. For example, the effector function of a dendritic cell can be increased survival, differentiation, phagocytosis, and/or antigen cross-presentation. Thus, the term “signal transducing domain” refers to the portion of a protein which transduces the effector signal function signal and directs the cell to perform a specialized function.

Various intracellular domains have different functions in different cell types. The present disclosure provides for an intracellular signaling domain useful in DCs. As described herein, Fc receptor-based, toll-like receptor (TLR)-based, or FMS-like tyrosine kinase 3 (Flt3)-based IC domains were directly compared, and the Flt3-based IC domains were discovered to be the most effective at generating functional CAR-DCs.

As described herein, the intracellular domain can be an FMS-like tyrosine kinase 3 (Flt3) intracellular domain. The Flt3 signaling domain is encoded by the Flt3 gene. Flt3 encodes a class III receptor tyrosine kinase that acts as a receptor for the cytokine Flt3 ligand (Flt3L). The intracellular domain derived from Flt3 may be critical for successfully achieving CAR dendritic cells. In one embodiment, the CAR comprises an intracellular signaling domain comprising at least 50% sequence identity, at least 60% sequence identity, at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to the intracellular domain from the protein Flt3, which may be mouse or human. FMS-like tyrosine kinase 3 (FLT-3) (also known as cluster of differentiation antigen 135 (CD135), receptor-type tyrosine-protein kinase Flt3, or fetal liver kinase-2 (Flk2)) is a protein that in humans is encoded by the FLT3 gene. Flt3 is a cytokine receptor which belongs to the receptor tyrosine kinase class III. Flt3 is the receptor for the cytokine Flt3 ligand (FLT3L). Flt3 is composed of five extracellular immunoglobulin-like domains, an extracellular domain, a transmembrane domain, a juxtamembrane domain and a tyrosine-kinase domain consisting of 2 lobes that are connected by a tyrosine-kinase insert. Cytoplasmic Flt3 undergoes glycosylation, which promotes localization of the receptor to the membrane. The nucleic acid sequences and peptide sequences of Flt3 can be found in publicly available databases, including, for example Entrez gene accession number 2322 and UniProt accession number P36888.

Flt3 tyrosine-protein kinase that acts as cell-surface receptor for the cytokine FLT3L and regulates differentiation, proliferation and survival of hematopoietic progenitor cells and of dendritic cells. Flt3 promotes phosphorylation of SHC1 and AKT1, and activation of the downstream effector MTOR. It promotes activation of RAS signaling and phosphorylation of downstream kinases, including MAPK1/ERK2 and/or MAPK3/ERK1. It also has been shown to promote phosphorylation of FES, FER, PTPN6/SHP, PTPN11/SHP-2, PLCG1, and STAT5A and/or STAT5B. Activation of wild-type FLT3 causes only marginal activation of STAT5A or STAT5B.

The FMS-like tyrosine kinase 3 (Flt3)-based IC domain can be any Flt3-based or Flt3-derived IC domain, such as active variants or functional fragments of the human Flt3 IC domain, which may have the amino acid sequence set forth in SEQ ID NO: 6. In some aspects, the intracellular domain may comprise an amino acid sequence at least about at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence set forth in SEQ ID NO: 6, or a functional variant, derivative, or fragment thereof. In another example, the Flt3 IC domain may be from mouse and may be at least about at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence set forth in SEQ ID NO: 5, or a functional variant, derivative, or fragment thereof.

In one embodiment, the CAR has the following structure: signal peptide—antigen binding domain (with a linker sequence connecting the variable regions)—extracellular domain—transmembrane domain—Flt3 intracellular domain.

In some embodiments, the CAR may have the properties of different intracellular domains by including two or more intracellular domains. For example, such combinations can include one intracellular domain from the Flt3 family and one intracellular domain from an ITAM domain-containing protein or a TIR-domain containing protein, resulting in the simultaneous activation of different signaling pathways. These are considered costimulatory domains, described in more detail above.

Each intracellular domain can have unique properties. Differences in the affinity of the scFv, the intensity of antigen expression, the probability of off-tumor toxicity, or the disease to be treated may influence the selection of the intracellular domain.

The CAR construct moieties or components can be operably linked with a linker. A linker can be any nucleotide sequence capable of linking the moieties described herein. For example, the linker can be any amino acid sequence suitable for this purpose (e.g., of a length of 8-80 amino acids, depending on the target-binding domain being used). In some aspects, the linker is a molecule that is used to connect the variable domains of the heavy (V_{H}) and light chains (V_{L}) with their respective non-variable domains of immunoglobulins to construct a single chain antibody (scFv), or to engineer bivalent single chain variable fragments (bi-scFvs) by linking two scFvs. In some aspects, any suitable linker can be incorporated into the CAR of the current disclosure. In an exemplary aspect, the linker sequence of the CAR may comprise an amino acid sequence at least about 80% identical to SEQ ID. NO: 16.

In some aspects, the current disclosure encompasses a CAR comprising an extracellular domain no longer than 40 amino acids in length and an intracellular domain from an FMS-like tyrosine kinase 3 (Flt3) intracellular domain. In some aspects the CAR comprises an extracellular domain of 5-20 amino acids in length, for example 5, 6, 7, 8, 9, 10, 11, 12, 13, 14, 15, 16, 17, 18, 19, 20, 21, 22, 23, 24, 25, 26, 27, 28, 29, or 30 amino acids in length and an intracellular domain having at least about 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of an FMS-like tyrosine kinase 3 (Flt3) intracellular domain. In some aspects the extracellular domain comprises a portion of a CD8 extracellular domain. In some aspects, the CAR comprises an extracellular domain comprising no more than 5, 6, 7, 8, 9, 10, 11, 12, 13, 14, 15, 16, 17, 18, 19, 20 20, 21, 22, 23, 24, 25, 26, 27, 28, 29, or 30 consecutive amino acids of a mammalian (for example, mouse or human) CD8 extracellular domain and an intracellular domain having at least about 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of a mammalian FMS-like tyrosine kinase 3 (Flt3) intracellular domain (for example, mouse or human). In some aspects the CAR comprises a CD8 extracellular domain comprising a polypeptide sequence at least about 80% identical to SEQ ID NO: 7, 8 or 18 and the intracellular domain comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NOS: 5, or 6 or a fragment, variant or derivative thereof.

In one embodiment, the CAR has the following structure: signal peptide—antigen binding domain (with a linker sequence connecting the variable regions)—extracellular domain<13 amino acids in length—transmembrane domain—Flt3 intracellular domain.

In some aspects, the current disclosure encompasses a CAR comprising from N-terminus to C-terminus, a signal peptide as disclosed herein, an antigen binding domain as disclosed herein, an extracellular domain no longer than 40 amino acids and as disclosed herein, a transmembrane domain as disclosed herein and an intracellular signaling domain comprising an FMS-like tyrosine kinase 3 (Flt3) signaling domain or a functional fragment, variant or derivative thereof.

In an exemplary aspect, the current disclosure encompasses a mouse CAR comprising an (a) extracellular domain comprising the amino acid sequence set forth in SEQ ID NO: 7 or 18, (b) a mouse transmembrane domain comprising the amino acid sequence set forth in SEQ ID NO: 11 or 19 and (c) an intracellular domain comprising the amino acid sequence set forth in SEQ ID NO: 5 or a fragment, variant or derivative thereof.

In an exemplary aspect, the current disclosure encompasses a human CAR comprising an (a) extracellular domain comprising an amino acid sequence set forth in SEQ ID NO: 7 or 18, (b) a human transmembrane domain comprising an amino acid sequence set forth in SEQ ID NO: 12 or 20 and (c) an intracellular domain comprising an amino acid sequence set forth in SEQ ID NO: 6.

In some aspects, the CAR as disclosed herein may be linked to suitable delivery agent for cellular and/or in vivo delivery. In some aspects, the CARs described herein may be linked to any delivery agent known in the art. The type of agent used depends on the type of applications envisaged. Such conjugates may be linked to substrates (e.g. chemicals, nanoparticles) and may be used e.g. to deliver therapy to a target of interest.

In some aspect, the agent is a nanoparticle. In some aspects, the nanoparticle may be composed of any core composed of metal, lipid, polymer, or biological macromolecule. In some aspects, the nanoparticle comprises a liposome, a protein nanogel, a polymer nanoparticle, or a solid nanoparticle. In embodiments, the nanoparticle, optionally, comprises at least one polymer, cationic polymer, or cationic block co-polymer on the particle surface. In other embodiments, the nanoparticle comprises a nanogel that is cross linked by a reversible linker that is sensitive to redox (disulfide) or pH (hydrolysable groups) or enzymes (proteases). In some aspects, the nanoparticle may be a magnetic nanoparticle, a controlled release polymer nanoparticle or lipid composition. Magnetic nanoparticles include, but are not limited to iron (e.g., Fe₃O₄ or Fe₂O₄), cobalt, zinc, cadmium, nickel, gadolinium, chromium, copper, manganese, terbium, europium, gold, silver, platinum, or alloys thereof. Controlled release polymer nanoparticles can be produced using conventional methods from biodegradable or nonbiodegradable polymers, e.g., poly(lactic acid), derivatives of poly(lactic acid), PEGylated poly(lactic acid), poly(lactic-co-glycolic acid), derivatives of poly(lactic-co-glycolic acid), PEGylated poly(lactic-co-glycolic acid), a polyanhydride, poly(ortho esters), derivatives of poly(ortho esters), PEGylated poly(ortho esters), poly(caprolactone), derivatives of poly(caprolactone), PEGylated poly(caprolactone), poly(acrylic acid), derivatives of poly(acrylic acid), poly(urethane), derivatives of poly(urethane), or combinations thereof). Similarly, lipid composition (e.g., liposomes, solid lipid nanoparticles and the like) can be produced using conventional methods and conjugated to an antibody of this invention.

A nanoparticle for use in the current disclosure may range in size from 1-1000 nm, 1-500 nm, 1-250 nm, 25-200 nm, 25-100 nm, 35-75 nm, or 25-60 nm. In some aspects, the nanoparticle is a lipid nanoparticle (LNP). In some aspects, the LNP has a diameter less than 1000 nm, 500 nm, 250 nm, 200 nm, 150 nm, 100 nm, 75 nm, 50 nm, or 25 nm. In some aspect, the agent is a solid lipid nanoparticle.

In some aspects, the CAR-containing cells of the present disclosure may comprise one or more distinct CAR constructs. For example, a dual CAR-containing cell may be generated by cloning a protein encoding sequence of a first antigen binding domain into a viral vector containing one or more costimulatory domains and a signaling transducing domain and cloning a second protein encoding sequence of a second antigen binding domain into the same viral vector containing an additional one or more costimulatory domains and a signaling transducing domain resulting in a plasmid from which the two CAR constructs are expressed from the same vector. A tandem CAR-containing cell is a cell with a single chimeric antigen polypeptide comprising two distinct antigen binding domains capable of interacting with two different cell surface molecules, wherein the antigen binding domains are linked together by a flexible linker and share one or more costimulatory domains, wherein the binding of the first or the second antigen binding domain will signal through one or more the costimulatory domains and a signaling transducing domain.

Genetic modification of a CAR-containing cell or progenitor thereof can be accomplished by transducing a substantially homogeneous cell composition with a recombinant DNA construct. In some aspects, the current disclosure also encompasses a polynucleotide sequence encoding a chimeric antigen receptor (CAR) as disclosed herein. In some aspects the polynucleotide sequence encoding the CAR comprises a nucleic acid sequence encoding a CD8 extracellular domain with a polypeptide sequence at least about 80% identical to SEQ ID NO: 7, 8, or 18 and a nucleic acid encoding an intracellular domain comprising an amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 5, or 6, or a fragment, derivative or variant thereof.

In certain embodiments, a retroviral vector (either gamma-retroviral or lentiviral) is employed for the introduction of the DNA construct into the cell. For example, a polynucleotide encoding a CAR can be cloned into a retroviral vector and expression can be driven from its endogenous promoter, from the retroviral long terminal repeat, or from a promoter specific for a target cell type of interest. Other viral vectors, or non-viral vectors may be used as well.

For initial genetic modification of a CAR-containing cell to include a CAR, a retroviral vector is generally employed for transduction, however any other suitable viral vector or non-viral delivery system can be used. The CAR can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB TRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, any vector or CAR disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nat. Acad. Sci. USA 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J Clin. Invest. 89:1817.

Other transducing viral vectors can be used to modify a CAR-containing cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a CAR-containing cell. For example, a nucleic acid molecule can be introduced into a CAR-containing cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to forma single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some aspects, the polynucleotide sequences, construct and complexes for transfection into a cell as disclosed herein may be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations. Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like. Polynucleotides may be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. In some aspects, the delivery particle is a nanoparticle. A nanoparticle for use in the current disclosure may range in size from 1-1000 nm, 1-500 nm, 1-250 nm, 25-200 nm, 25-100 nm, 35-75 nm, or 25-60 nm. In some aspects, the nanoparticle is a lipid nanoparticle (LNP). In some aspects, the LNP has a diameter less than 1000 nm, 500 nm, 250 nm, 200 nm, 150 nm, 100 nm, 75 nm, 50 nm, or 25 nm. In some aspects, such nanoparticles may be designed using a core composed of metal, lipid, polymer, or biological macromolecule, and multiple copies of the nucleic acid molecules can be attached to or encapsulated with the nanoparticle core. This increases the copy number of the DNA that is delivered to each cell and, so, increases the intracellular expression to maximize the likelihood that the encoded products will be expressed. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload. Nanoparticles may additionally be advantageously coupled to targeting molecules to direct the nanoparticle to the appropriate cell type and/or increase the likelihood of cellular uptake. Examples of such targeting molecules include antibodies specific for cell surface receptors and the natural ligands (or portions of the natural ligands) for cell surface receptors.

The resulting cells can be grown under conditions similar to those for unmodified cells, whereby the modified cells can be expanded and used for a variety of purposes.

Any targeted genome editing methods can be used to place presently disclosed CARs at one or more endogenous gene loci of a presently disclosed immunoresponsive cell. In certain embodiments, a CRISPR system is used to deliver presently disclosed CARs to one or more endogenous gene loci of a presently disclosed immunoresponsive cell. In certain embodiments, zinc-finger nucleases are used to deliver presently disclosed CARs to one or more endogenous gene loci of a presently disclosed immunoresponsive cell. In certain embodiments, a TALEN system is used to deliver presently disclosed CARs to one or more endogenous gene loci of a presently disclosed immunoresponsive cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Placement of a presently disclosed CAR can be made at any endogenous gene locus.

The present disclosure also provides pharmaceutical compositions. The pharmaceutical composition comprises a plurality of CAR-containing cells such as CAR-DCs, as an active component, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

Compositions comprising the presently disclosed CAR-containing cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the CAR-containing cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the CAR-containing cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10³ and about 10¹⁰, between about 10⁵ and about 10⁹, or between about 10⁶ and about 10⁸ of the presently disclosed CAR-containing cell are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed CAR-containing cells are administered to a human subject. In certain embodiments, between about 1×10⁷ and 5×10⁸ of the presently disclosed CAR-containing cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Compositions comprising the presently disclosed CAR-containing cells can be provided systemically or directly to a subject for inducing and/or enhancing an immune response to an antigen and/or treating and/or preventing a neoplasm, pathogen infection, or infectious disease. In certain embodiments, the presently disclosed CAR-containing cells or compositions comprising thereof are directly injected into a tumor or organ of interest (e.g., an organ affected by a neoplasia). Alternatively, the presently disclosed CAR-containing cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed CAR-DCs can be administered in any physiologically acceptable vehicle, normally intravascularly, although they may also be introduced into bone or other convenient site where the cells may find an appropriate site for regeneration and differentiation (e.g., lymphatics). Usually, at least a population of about 1×10⁵ cells will be administered. The presently disclosed CAR-containing cells can comprise a purified population of cells. Those skilled in the art can readily determine the percentage of the presently CAR-containing cells in a population using various well-known methods, such as fluorescence activated cell sorting (FACS). Suitable ranges of purity in populations comprising the presently disclosed CAR-containing cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed CAR-containing cells or their progenitors and a pharmaceutically acceptable carrier. Administration can be autologous or heterologous. For example, CAR-DCs, or progenitors can be obtained from one subject, and administered to the same subject or a different, compatible subject. Peripheral blood derived CAR-DCs or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed CAR-DCs), it can be formulated in a unit dosage injectable form (solution, suspension, emulsion).

### II. Methods

Cells disclosed herein, and/or generated using the methods disclosed herein, may be used in immunotherapy and adoptive cell transfer, for the treatment, or the manufacture of a medicament for treatment, of cancers, autoimmune diseases, infectious diseases, and other conditions. One aspect of the present disclosure provides for modified dendritic cells that stimulate an adaptive antitumor T cell response.

As described herein, the adaptive antitumor T cell response can be initiated or enhanced by antigen cross-presentation or cross-priming or cross-dressing from the CAR-DCs. Cross-presentation describes the process in which the modified dendritic cells take up, process, and present antigens (e.g., a tumor cell antigen) on the surface of the cell on a complex with a MHC I molecule. The antigen is then recognized by a T cell. Cross-priming describes the process in which recognition of the antigen by the T cell results in the T cell becoming activated. The activated T cell is then capable of enhanced proliferation, persistence, and/or targeted, enhanced cytotoxicity towards tumor cells expressing that antigen. Cross-dressing involves the transfer of preformed functional peptide-MHC complexes from the surface of donor cells to recipient cells, such as dendritic cells (DCs). These cross-dressed cells might eventually present the intact, unprocessed peptide-MHC complexes to T lymphocytes. In some aspects, the adaptive antitumor T cell response produced by CAR-DCs as described herein is due to cross-presentation. In some aspects, the adaptive antitumor T cell response produced by CAR-DCs as described herein is not due to cross-dressing.

As described herein, the adaptive antitumor T cell response can comprise, in a non-limiting example, an increase in T cell function. For example, T cell function can be assessed by the cytotoxic T cell lymphocyte assay (CTL), where an escalating ratio of effector T cells is mixed with target tumor cells for a defined amount of time (generally 4 hours), and tumor cell killing is quantified by tumor luciferase activity.

As described herein, the adaptive antitumor T cell response can also comprise an increase in T cell activation or proliferation. For example, T cell activation or proliferation can be measured by assessing CD4 and CD8 T cell division by FACS analysis for proliferation or for markers of activation, such as cytokine release.

As described herein, a successful adaptive antitumor T cell response can result in tumor cell cytotoxicity, further tumor cell phagocytosis, and reduction in tumor volume. The antitumor T cell response can directly eliminate antigen positive (Ag+) tumors targeted by the CARs, and indirectly eliminate CAR-Ag− tumor cells (not recognized directly by the CAR), through cross-presentation and epitope spreading. Epitope spreading refers to the broadening of the immune response to include T cell and antibody specificities beyond the antigen that originally triggered the immune response. For example, epitope spreading can result in tumor cells that do not express the antigen targeted by the CAR to be targeted by T cells.

Thus, the present disclosure provides a method of stimulating an adaptive antitumor T cell response in a subject, wherein the method generally comprises administering an effective amount of CAR-containing cells to the subject. The CAR-containing cells targets a tumor or cancer cell, phagocytizes the tumor or cancer cell and cross-presents tumor antigens to the subject's T cells. Accordingly, the CAR-containing cells directly target antigen positive (Ag⁺) tumor or cancer cells for elimination and/or indirectly targets CAR-antigen negative (Ag⁻) tumor or cancer cells for elimination through cross-presentation and epitope spreading.

In another embodiment, the present disclosure provides methods for reducing or preventing cancer recurrence in a subject, wherein the method generally comprises administering an effective amount of CAR-containing cells to the subject, which target an antigen expressed by the cancer or tumor cell. A recurrence occurs when the cancer comes back after the initial treatment. This can happen weeks, months, or even years after the primary or original cancer was treated. As described herein, the present disclosure is shown to produce a lasting adaptive antitumor T cell response, see, e.g., Example 1(vi).

In some embodiments, the present disclosure provides methods for treating a cancer or tumor in a subject, wherein the method generally comprises administering CAR-containing cells or CAR-comprising agents to the subject, which target an antigen expressed by the cancer or tumor cell. An effective of amount of CAR-containing cells may be administered. This treatment can be particularly efficacious for solid tumors, but may be directed against any form of cancer. Traditional chimeric antigen receptor (CAR) T cells exhibit only a 1% complete response in solid tumors in clinical trials thus far. Solid tumors escape CAR T recognition if not all cells express the target antigen. Successfully creating an adaptive immune response in patients would overcome the failures of both types of immunotherapy. Dendritic cells (DCs) are critical in initiating an adaptive immune response. CAR-containing cells enable new therapeutic strategies to directly eliminate antigen positive (Ag+) tumors targeted by the CARs, and indirectly eliminate Ag− solid tumor cells (not recognized by the CAR), through epitope spreading.

In some aspects, the present disclosure provides methods for treating a cancer or tumor in a subject, wherein the method comprises administering CAR-comprising agents to the subject, which target an antigen expressed by the cancer or tumor cell. In some aspects, the CAR-comprising agent is a nanoparticle as disclosed herein.

A tumor or cancer can be any tumor or cancer that occurs in (or is a metastatic cancer originating from) the bladder, breast, bone, cervix, muscle, brain and nervous system, endocrine system, endometrium, eye, lip, oral, liver, lung, gastrointestinal system (e.g., colon, rectal), genitourinary and gynecologic systems (e.g., cervix, ovary), head and neck, hematopoietic system, kidney, skin, pancreas, prostate, thyroid, bone, thoracic and respiratory system, or any other human tissue that has undergone a malignant transformation. A solid tumor is one derived from any human cell other blood cells. In some aspects, a solid tumor may be a sarcoma, carcinoma, or lymphoma.

The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL)Sezary syndrome, and others as defined herein and known in the art.

Leukemias include Acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

In some embodiments, the medicament can be used for treating cancer in a patient, particularly for the treatment of solid tumors such as melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

In another embodiment, the medicament can be used for treating cancer in a patient, particularly for the treatment of hematologic malignancies selected from multiple myeloma and acute myeloid leukemia (AML) and for T-cell malignancies selected from T-cell acute lymphoblastic leukemia (T-ALL), non-Hodgkin's lymphoma, and T-cell chronic lymphocytic leukemia (T-CLL).

Non-limiting examples of neoplasms or cancers that may be treated with a method of the disclosure may include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-cell lymphoma (cutaneous), T-cell leukemia and lymphoma, testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenstrom macroglobulinemia, or Wilms tumor (childhood).

In some aspects, the current disclosure encompasses methods of treatments for solid tumor. In some aspects, a solid tumor may be a sarcoma, carcinoma, or lymphoma. In some exemplary aspects, the solid tumor that can be treated with the methods disclosed herein expresses a tumor antigen selected from a group consisting of EphA2, alphafetoprotein (AFP), carcinoembryonic antigen (CEA), B7H3, CA-125, MUC-1 antibody, CD19, CD20, CD123, CD22, CD30, SlamF7, CD33, EGFRvIII, BCMA, GD2, CD38, PSMA, B7H3, EPCAM, IL-13Ra2, PSCA, Mesothelin, Her2, LewisY, LewisA, CIAX, epithelial tumor antigen (ETA), tyrosinase, melanoma-associated antigen (MAGE), abnormal products of ras or p53, or other proteins found to be more highly enriched on the surface of tumor cells than critical normal tissues. In some exemplary aspects, the solid tumor expresses a tumor antigen selected from a group consisting of CD19, EphA2 and EGFRvIII. Examples of tumors expressing EphA2 tumor antigen include prostate, lung, esophageal, colorectal, cervical, ovarian, breast and skin cancers. Non-limiting examples of tumors expressing EGFRvIII include gliomas of the brain, colorectal cancers, esophageal cancers, lung, prostate cancers, ovarian cancers, and sarcomas. Non-limiting examples of tumors expressing CD19 antigen include acute lymphoblastic leukemias, chronic lymphocytic leukemias and B cell lymphomas.

Thus, aspects of the present disclosure is a method for treating a subject in need thereof. The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

Also provided is a process of treating or preventing a proliferative disease, disorder, or condition (e.g., a tumor or cancer, or metastases thereof) in a subject in need of administration of a therapeutically effective amount of a dendritic cell-based therapy as described herein so as to reduce or eliminate the tumor or cancer.

Methods described herein are generally performed on a subject in need thereof. A subject in need of the therapeutic methods described herein can be a subject having, diagnosed with, suspected of having, or at risk for developing a cancer or proliferative disease, disorder, or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of CAR-DC therapy is, for example, that amount that would cause the desired therapeutic effect in a subject while minimizing undesired side effects. In various embodiments, an effective amount of a dendritic cell-based therapy described herein can substantially inhibit tumor growth or cancer progression, slow the progress of a tumor or cancer, or limit the development of a tumor or cancer.

When used in the treatments described herein, a therapeutically effective amount of a CAR-DC therapy can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce or cure a proliferative disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of CAR-DC therapy can occur as a single event or over a time course of treatment. For example, dendritic cell-based therapy can be administered daily, weekly, bi-weekly, or monthly. For more chronic conditions, treatment could extend from several weeks to several months or years.

Treatment in accord with the methods described herein can be performed prior to, concurrent with, or after conventional treatment modalities for cancer or proliferative disease, disorder, or condition.

A CAR therapy can be administered simultaneously or sequentially with another agent, such as an anti-cancer therapy, or another agent. For example, a dendritic cell-based therapy can be administered before, after, or simultaneously with another agent, such as a chemotherapeutic agent, another form of immune therapy, or radiation therapy. Simultaneous administration can occur through the administration of separate compositions, each containing one or more of a dendritic cell-based therapy and another agent, such as a chemotherapeutic agent, additional immune therapy, or radiation therapy. Simultaneous administration can occur through the administration of one composition containing two or more of a dendritic cell-based therapy, an antibiotic, an anti-inflammatory, or another agent, such as a chemotherapeutic agent, immune therapy, or radiation therapy.

The administration of CAR-containing cells or a population of CAR-containing cells or CAR-comprising agents of the present disclosure may be carried out by aerosol inhalation, injection, ingestion, transfusion, implantation or transplantation. The compositions described herein, may be administered to a patient through one or more of a subcutaneous, intradermal, a parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, or and transdermal route intranodal, intramedulla, intramuscular, by intravenous or intralymphatic injection, or intraperitoneal routes. In one embodiment, the cell compositions of the present disclosure are preferably administered by intravenous injection.

In some aspects, the CAR-containing cells or a population of CAR-containing cells or CAR comprising delivery vehicles can be administrated in one or more doses. In one aspect, the effective amount of CAR-containing cells or a population of CAR-containing cells or CAR-comprising agents are administrated as a single dose. In another embodiment, the effective amount of cells or agents are administered as more than one dose over a period time. In some aspects, the cells can be administered one, or two, or three, or four, or five, or six or more times. In some aspects, the cells may be administered on a daily, a weekly, or bi-weekly, or once every 3 weeks, or monthly or less frequency during the course of the treatment. In some exemplary aspects, the CAR-containing cells can be administered in three injections, spaced 4 weeks apart, delivered intravenously. In some aspects, the number of CAR-containing cells per injection can be between about 1×10⁹ to about 1×10⁶ or any number in between. In some aspects, the number of cells can be at least about 1×10⁶, or at least about 5×10⁶, or at least about 10×10⁶, or at least about 20×10⁶, or at least about 30×10⁶, or at least about 40×10⁶, or at least 50×10⁶, or at least about 60×10⁶, or at least about 70×10⁶, or at least about 80×10⁶, or at least about 90×10⁶, or at least about 100×10⁶. Timing of administration is within the judgment of a health care provider and depends on the clinical condition of the patient. The CAR-containing cells or a population of CAR-containing cells may be obtained from any source, such as a blood bank or a donor. While the needs of a patient vary, determination of optimal ranges of effective amounts of a given CAR-containing cells population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

In another embodiment, the effective amount of CAR-containing cells or a population of CAR-containing cells or CAR-comprising agents or composition comprising these are administered parenterally. The administration can be an intravenous administration. The administration of CAR-containing cells or a population of CAR-containing cells or composition comprising those CAR-containing cells can be directly done by injection within a tumor.

In one embodiment of the present disclosure, the CAR-containing cells or a population of the CAR-containing cells or CAR-comprising agents are administered to a patient in conjunction with, e.g., before, simultaneously or following, any number of relevant treatment modalities, including but not limited to, treatment with cytokines, or expression of cytokines from within the CAR-containing cells, that enhance dendritic cell or T-cell proliferation and persistence and, include but not limited to, Flt3L, IL-2, IL-7, and IL-15 or analogues thereof.

In some embodiments, the CAR-containing cells or a population of CAR-containing cells or CAR-comprising agents of the present disclosure may be used in combination with agents that inhibit immunosuppressive pathways, including but not limited to, inhibitors of TGFβ, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2.

In another embodiment, the CAR-containing cells or a population of CAR-containing cells or CAR-comprising agent of the present disclosure may be used in combination with T-cell checkpoint inhibitors, including but not limited to, anti-CTLA4 (such as Ipilimumab) anti-PD1 (such as Pembrolizumab, Nivolumab, Cemiplimab), anti-PDL1 (such as Atezolizumab, Avelumab, Durvalumab), anti-PDL2, anti-BTLA, anti-LAG3, anti-TIM3, anti-VISTA, anti-TIGIT, and anti-KIR.

In another embodiment, the CAR-containing cells or a population of CAR-containing cells or CAR-comprising agent of the present disclosure may be used in combination with T cell agonists, including but not limited to, antibodies that stimulate CD28, ICOS, OX-40, CD27, 4-1BB, CD137, GITR, and HVEM.

In another embodiment, the CAR-containing cells or a population of CAR-containing cells or CAR-comprising agent of the present disclosure may be used in combination with therapeutic oncolytic viruses, including but not limited to, retroviruses, picornaviruses, rhabdoviruses, paramyxoviruses, reoviruses, parvoviruses, adenoviruses, herpesviruses, and poxviruses.

In another embodiment, the CAR-containing cells or a population of CAR-containing cells or CAR-comprising agent of the present disclosure may be used in combination with immunostimulatory therapies, such as toll-like receptors agonists, including but not limited to, TLR3, TLR4, TLR7 and TLR9 agonists.

In another embodiment, the CAR-containing cells or a population of CAR-containing cells or CAR-comprising agent of the present disclosure may be used in combination with stimulator of interferon gene (STING) agonists, such as cyclic GMP-AMP synthase (cGAS).

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to DC cells, DC progenitors, DC precursors, or modified cells thereof, CAR constructs, or CAR-DC cells or a nucleic acid sequence encoding a CAR construct, and delivery systems. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

Described herein is a method of generating chimeric antigen receptor dendritic cells (CAR-DCs).

Precursor cells, for example, stem cells, monocytes, or in this case bone marrow cells were isolated, grown in Flt3L for about one day, then virally transduced with a CAR of interest, then further differentiated with Flt3L for about 2-15 days to generate DC-like cells for use in vivo or in vitro. CAR expression can be assessed using an antibody that recognizes the CAR on the cell surface by FACS analysis. Viral transduction was done here using retrovirus or lentivirus, but may be achieved with any gene delivery method.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription. In some aspects, the promoter in the current context is a sequence that can drive the expression of a gene in dendritic cells. In some aspects, the promoter is a CD11c promoter or a variant or derivative thereof. In some aspects, the promotor comprises a nucleic acid sequence at least about 60% identical to any one of SEQ ID NO:1, SEQ ID NO:2, or SEQ ID NO:3. In some aspects, the promoter comprises a nucleic acid sequence at least about 60%-70%, or 70%-80%, or 80%-90%, or 90%-100^{%}identical to any one of SEQ ID NO:1, SEQ ID NO:2, or SEQ ID NO:3.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

The terms “derivative,” “variant,” “variations” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants, “variations” and fragments of a polypeptide can comprise one or more amino acid variations (e.g., mutations, insertions, and deletions), truncations, modifications, or combinations thereof compared to a wild type polypeptide. A part or fragment of a polypeptide may correspond to at least 1%, at least 2%, at least 3%, at least 4%, at least 5%, at least 10%, at least 20%, at least 30%, at least 40% of the length of a polypeptide, such as a polypeptide having an amino acid sequence identified by a specific SEQ ID NO:, or having at least 50%, or at least 60%, or at least 70%, or at least 80%, or at least 90% of the length (in amino acids) of the polypeptide.

Within the context of the present application, a protein is represented by an amino acid sequence, and correspondingly a nucleic acid molecule or a polynucleotide is represented by a nucleic acid sequence. Identity and similarity between sequences: throughout this application, it should be understood that for each reference to a specific amino acid sequence using a unique sequence identifier (SEQ ID NO:), the sequence may be replaced by: a polypeptide represented by an amino acid sequence comprising a sequence that has at least 60% sequence identity or similarity with the reference amino acid sequence. Another level of sequence identity or similarity is 65%. Another level of sequence identity or similarity is 70%. Another level of sequence identity or similarity is 75%. Another level of sequence identity or similarity is 80%. Another level of sequence identity or similarity is 85%. Another level of sequence identity or similarity is 90%. Another level of sequence identity or similarity is 95%. Another level of sequence identity or similarity is 98%. Another level of sequence identity or similarity is 99%.

Each nucleic acid or amino acid sequence described herein by virtue of its identity or similarity percentage with a given amino acid sequence respectively has in a further aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In an aspect, sequence identity is calculated based on the full length (in amino acids or nucleotides) of given SEQ ID NO: or based on a portion thereof. A portion of a full-length sequence may be referred to as a fragment, and may mean at least 50%, 60%, 70%, 80%, 90%, or 100% of the length (in amino acids or nucleotides) of a reference sequence. “Identity” also refers to the degree of sequence relatedness between two amino acid sequences, or between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined, for example, by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below:

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu |
| | (L), and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P |
| | and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 50-99% identity to the reference sequence described herein and screen such for desired phenotypes according to methods routine in the art.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° cin the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log 10[Na+])+0.41(fraction G/C content)−0.63(% formamide)−(600/1). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

The term “activation” (and other conjugations thereof) in reference to cells is generally understood to be synonymous with “stimulating” and as used herein refers to an enhanced functional outcome and/or expansion of cell populations.

The term “antigen” as used herein is a molecule or molecular structure that an antigen receptor or an antigen-binding protein can recognize (for example, bind to). An antigen can be or can comprise, for example, a peptide, a polypeptide, a carbohydrate, a chemical, a moiety, a non-peptide antigen, a phosphoantigen, a tumor-associated antigen, a neoantigen, a tumor microenvironment antigen, a microbial antigen, a viral antigen, a bacterial antigen, an autoantigen, a glycan-based antigen, a peptide-based antigen, a lipid-based antigen, or any combination thereof. In some aspects, an antigen is capable of inducing an immune response. In some examples, an antigen binds to an antigen receptor or antigen-binding protein, or induces an immune response, when present in a complex e.g., presented by MHC. In some cases, an antigen adopts a certain conformation in order to bind to an antigen receptor or antigen-binding protein, and/or to induce an immune response, e.g., adopts a conformation in response to the presence or absence of one or more metabolites. Antigen can refer to a whole target molecule, a whole complex, a or a fragment of a target molecule or complex that binds to an antigen receptor or an antigen-binding protein. Antigen receptors that recognize antigens include exogenous antigen-recognition receptors disclosed herein and other antigen-recognition receptors, such as endogenous T cell receptors. In the context of a CAR target, an antigen is a cell surface protein recognized by (i.e., that is the target of) chimeric antigen receptor. In the classical sense antigens are substances that are recognized by antibodies or the T-cell receptor, but the definitions overlap insofar as the CAR comprises antibody-derived domains such as light (V_{L}) and heavy (V_{H}) chains recognizing one or more antigen(s). An antigen can also comprise any intracellular or surface molecule, generally a protein or peptide, capable of being recognized by the immune system (most frequently T-cells, or antibodies).

The term “cancer” refers to a malignancy or abnormal growth of cells in the body. Many different cancers can be characterized or identified by particular cell surface proteins or molecules. Thus, in general terms, cancer in accordance with the present disclosure may refer to any malignancy that may be treated with an immune effector cell, such as a CAR-DCs as described herein, in which the modified dendritic cell recognizes and binds to the cell surface protein on the cancer cell. As used herein, cancer may refer to a hematologic malignancy, such as multiple myeloma, a T-cell malignancy, or a B cell malignancy. T cell malignancies may include, but are not limited to, T-cell acute lymphoblastic leukemia (T-ALL) or non-Hodgkin's lymphoma. A cancer may also refer to a solid tumor, such as including, but not limited to, cervical cancer, pancreatic cancer, ovarian cancer, mesothelioma, and lung cancer.

A “cell surface protein” as used herein is a protein (or protein complex) expressed by a cell at least in part on the surface of the cell. Examples of cell surface proteins include the TCR (and subunits thereof) and CD7.

A “chimeric antigen receptor” or “CAR” as used herein and generally used in the art, refers to a recombinant fusion protein that has an antigen binding domain, a transmembrane domain, and a signaling transducing domain that directs the cell to perform a specialized function upon binding of the antigen binding domain to a component present on the target cell. For example, a CAR can have an antibody-based specificity for a desired antigen (e.g., tumor antigen) with a T cell receptor-activating intracellular domain to generate a chimeric protein that exhibits specific anti-target cellular immune activity. First-generation CARs include an antigen binding domain and signaling transducing domain, commonly CD3ζ or FcεRIγ. Second generation CARs are built upon first generation CAR constructs by including an intracellular costimulatory domain, commonly 4-1BB or CD28. These costimulatory domains help enhance CAR-T cell cytotoxicity and proliferation compared to first generation CARs. The third generation CARs include multiple costimulatory domains, primarily to increase CAR-T cell proliferation and persistence. Chimeric antigen receptors are distinguished from other antigen binding agents by their ability both to bind MHC-independent antigens and transduce activation signals via their intracellular domain.

A “modified cell” refers to a cell in which the nuclear, organellar or extrachromosomal nucleic acid sequences of a cell has been transformed, modified or transduced using recombinant DNA technology to comprise a heterologous nucleic acid molecule, and is used interchangeably with “engineered cell,” “transformed cell,” and “transduced cell.” The cells are engineered, e.g., by the introduction of an exogenous nucleic acid sequence as defined herein. Such a cell has been genetically modified for example by the introduction of for example one or more mutations, insertions and/or deletions in the endogenous gene and/or insertion of a genetic construct in the genome. The modification may have been introduced using recombinant DNA technology. An engineered cell may refer to a cell in isolation or in culture. Modified cells may be “transduced cells” wherein the cells have been infected with e.g., a modified virus, for example, a retrovirus may be used but other suitable viruses may also be contemplated such as lentiviruses. Non-viral methods may also be used, such as transfections. Modified cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient but may also be a stable transfection wherein cells can be selected that have the gene construct integrated in their genome. In some cases, genetic engineering systems such as CRISPR or Argonaute may be utilized to design engineered cells that express a polypeptide described herein. In some aspects, the modified cell are dendritic cell or dendritic cell precursors engineered to comprise a construct encoding a CAR as disclosed herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems. A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

The term “composition” as used herein refers to an immunotherapeutic cell population combination with one or more therapeutically acceptable carriers.

“Pharmaceutical composition” means a mixture of substances suitable for administering to an individual that includes a pharmaceutical agent. As used herein a pharmaceutical composition comprises one or more of receptors, vectors, cells disclosed herein compounded with suitable pharmaceuticals carriers or excipients.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “patient”, “subject”, or “test subject” refers to any organism to which provided compound or compounds described herein are administered in accordance with the present invention e.g., for experimental, diagnostic, prophylactic, and/or therapeutic purposes. Typical subjects include animals (e.g., mammals such as mice, rats, rabbits, non-human primates, humans, insects etc.). In an aspect, a subject is a human. In some aspects, a subject may be suffering from, and/or susceptible to a disease, disorder, and/or condition (e.g., cancer). As used herein, a “patient population” or “population of subjects” refers to a plurality of patients or subjects.

The term “effective amount” as used herein is defined as the amount of the pharmaceutical composition of the present invention that are necessary to result in the desired physiological change in the cell or tissue to which it is administered. The term “therapeutically effective amount” as used herein is defined as the amount of pharmaceutical composition or number of cells of the present invention that achieves a desired effect with respect to cancer. In this context, a “desired effect” is synonymous with “an anti-tumor activity” as earlier defined herein. A skilled artisan readily recognizes that in many cases the pharmaceutical composition or cells may not provide a cure but may provide a partial benefit, such as alleviation or improvement of at least one symptom or parameter. In some embodiments, a physiological change having some benefit is also considered therapeutically beneficial. Thus, in some embodiments, an amount of pharmaceutical composition or number of cells that provides a physiological change is considered an “effective amount” or a “therapeutically effective amount.”

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The Examples below were conducted using the exemplary techniques provided herein for guidance, though equivalent techniques known to those skilled in the art can be used.

### Exemplary Techniques

### CAR Construction

CAR constructs were constructed using standard molecular biology techniques. The CAR consisted of a signal peptide (SEQ ID. NO: 9 or SEQ ID. NO: 10), scFv directed against the target listed, extracellular domain of defined length, transmembrane domain, and intracellular domain from Flt3. CAR mRNA was produced by in vitro transcription with the addition of a 5′ and 3′ UTR (untranslated region) and a polyA tail. The scFv targeting human CD19 was obtained from the previously published FMC63 antibody clone. The scFv targeting EGFRvIII was as previously published. Mouse CD8b extracellular extracellular domain domain was used, and for syngeneic mouse studies a codon optimized mouse Flt3 intracellular signaling domain was used (SEQ ID. NO: 5, the sequence of the human equivalent Flt3 intracellular signaling domain is provided as SEQ ID NO: 6). The mouse CD8 extracellular domain was designed in two different lengths: short (5 amino acids, SEQ ID NO: 7) and long (35 amino acids, SEQ ID NO: 8). A FLAG tag was added to the N-extracellular domain to allow easy detection. With the FLAG tag the length of the short extracellular domain was 13 amino acids and the long extracellular domain was 43 amino acids. The transmembrane domain was derived from mouse CD8b (SEQ ID NO: 11). Equivalent human transmembrane from CD8α (SEQ ID NO: 12) or CD8b (SEQ ID NO: 13).

### Isolation and Culture of BM Cells

Femurs and tibias of naïve Ly5.1 C57BL/6 mice were isolated and then flushed using a 10 ml syringe, 25 G needle, and PBS and filtered through a 70-μm strainer. Red blood cells (RBC) were lysed using ACK Lysis Buffer (Gibco). After RBC lysis, cells were brought up in complete Iscove's modified Dulbecco's medium (IMDM; Gibco) supplemented with 10% FBS (Gibco), 55 μmol/L 2-mercaptoethanol (Sigma-Aldrich), 1% penicillin/streptomycin solution (Gibco), 1 mmol/L sodium pyruvate (Corning), 1% MEM nonessential amino acid (Sigma-Aldrich), and 200 μmol/L glutamine (Gibco) and kept at 4° C. until plated for culture. Flt3L-cDCs were generated by plating BM cells at a density of 0.625 to 1.5×106/mL in complete IMDM supplemented with Flt3 ligand (Flt3L; 10% media produced by Chinese Hamster Ovary ie CHO cells stably secreting Flt3L, or 100 ng/ml recombinant Flt3L from Miltenyi) and stem cell factor (1% conditioned media or 100 ng/ml) for 4 days. On day 4 the cells were collected and spun at 1400 rpm for 10 minutes and then media was changed to complete IMDM supplemented with Flt3 ligand (10% conditioned media or 100 ng/ml) and cultured for an additional 7 days. Loosely adherent cells were isolated by gentle pipetting.

### Electroporation of CAR DCs

On day 9 of Flt3L differentiated BM cells, the cells were collected and counted using a hemocytometer. The cells were washed using OCS optimization buffer (Maxcyte) and then resuspended into a concentration of 500 mil/ml in OCS optimization buffer. The cells were split into three groups and the correct mRNA was added at a concentration of 100 μg/ml and then placed into one slot of an OC25×3 processing assembly (Maxcyte). The processing assembly was then electroporated using MaxCyte GT with DC1 protocol. The cells were then plated in complete IMDM supplemented with 10% Flt3L-cho at a concentration of 2.5 mil/ml. After 24 hours a portion of cells were assessed by spectral flow cytometry (Cytek Northern Lights) with the following scheme B220-CD11c+MHCII+XCR1+Sirpa-Thy1.1+ for CAR+ cDC1 and B220-CD11c+MHCII+XCR1-Sirpa+Thy1.1+ for CAR+ cDC2. The cells were then collected and washed 2 times using PBS and then resuspended in a final concentration of 16 mil/ml cDC1s in PBS.

### Tumor Cell Lines

The methylcholanthrene (MCA)-induced fibrosarcomas were generated in female C57Bl/6 mice, tested for Mycoplasma, and banked at low passage. The human CD19 and mRuby expressing fibrosarcoma (hCD19+ 1956) was generated by retrovirally transducing the 1956 cell lines with a plasmid encoding for extracellular human CD19 and H2A targeted mRuby and then sorted to ensure homogenous population. The human EGFRvIII and zsGreen expressing fibrosarcoma (EGFRvIII+ 1956) was generated by retrovirally transducing the 1956 cell lines with a plasmid encoding for the extracellular domain of mutant EGFRvIII and a cytoplasmic zsGreen and then sorted to ensure homogenous population. To generate a B2M⁻ 1956 cell line, the 1956 cells were transfected with a plasmid that encoded for a beta 2 microglobulin gRNA, an antibiotic resistance gene (puromycin), and CRISPR Cas9 to knockout B2M. These cells were cultured in puromycin for two days and subsequently clones were sorted for B2M negativity. These cells were then modified in the same way as the normal 1956 cell lines to generate hCD19+B2M⁻ 1956 cells and EGFRvIII⁺B2M⁻ 1956 cells.

### Cross Priming Experiments

OTI TCR transgenic (Jackson Laboratories) mouse spleens were harvested and dispersed into single-cell suspensions by mechanical separation. Naïve OTI T cells (ovalbumin specific T-cells) were isolated using naïve CD8 T cell isolation kit (Miltenyi Biotec) and then labeled with CellTrace™ Violet (CTV) (Invitrogen) and placed into a concentration of 0.5×10⁶ cells/ml in complete IMDM supplemented with 10% Flt3L-cho. hCD19+ or EGFRvIII+B2M-1956 cells were osmotically loaded with 10 mg/ml soluble ovalbumin (Worthington Biochemical Corporation) and then cultured at various concentrations (5×10⁶/ml, 2×10⁶/ml, 0.5×10⁶/ml) with 25,000 cDC1s and 25,000 CTV labeled naïve OT1 cells in a 96 well U-bottom. After 72 hours the proliferated OTI cells were analyzed via spectral flow cytometer (Cytek Northern lights) via CD45.1-mRuby-EGFRvIII-CD8a⁺TCRVa2+ and CTV dilution.

### Tumor Models

C57BL/6 WT mice were subcutaneously injected into the flanks with 106 hCD19+ 1956 tumor cells per 100 ul of PBS on both flanks. Tumor growth was measured every 2 to 3 days with a caliper, and tumor area was calculated by the multiplication of two perpendicular diameters. In accordance with our IACUC-approved protocol, maximal tumor diameter was 2 cm in one direction, and in no experiments was this limit exceeded. For intratumoral DC injections, Flt3L-cultured cDC1s were injected intratumorally one day post tumor implantation and tumor growth was measured as described above.

### Example 1: Development of Dendritic Cell CAR (CAR-DC) with FLT3 Receptor Signaling Domain

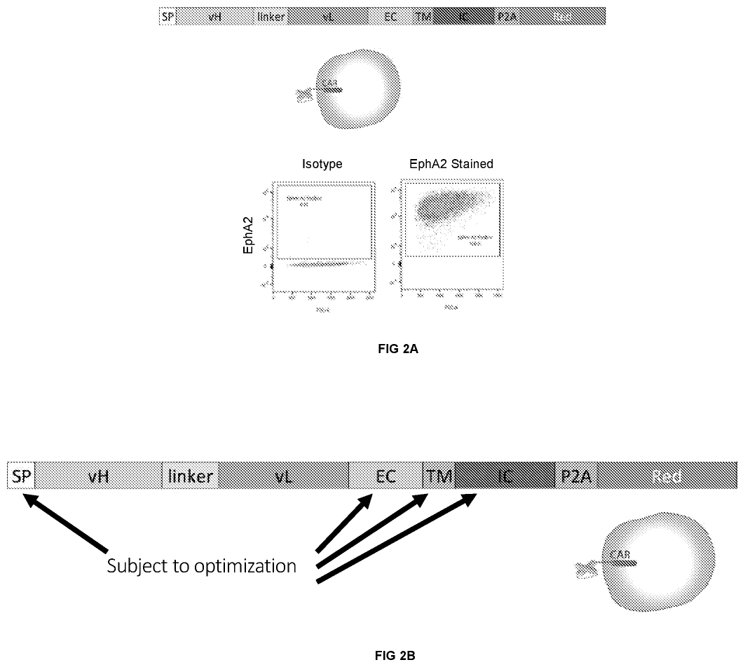

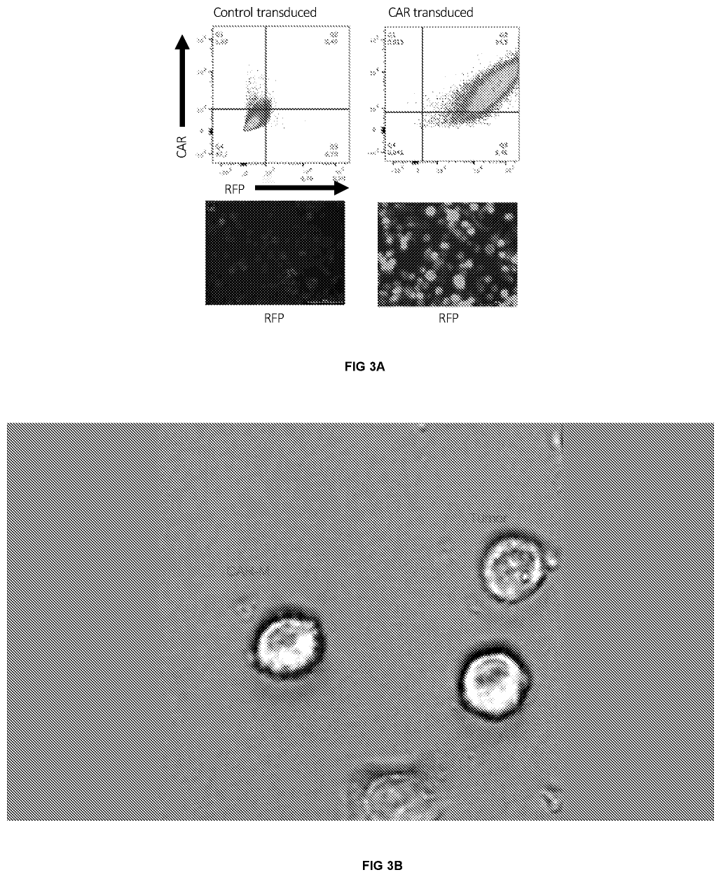

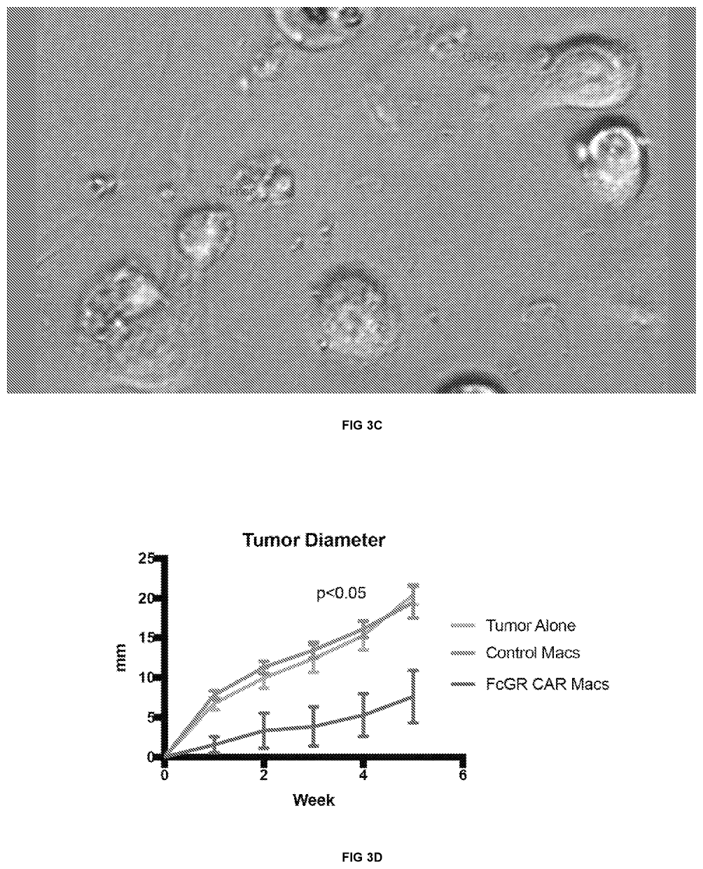

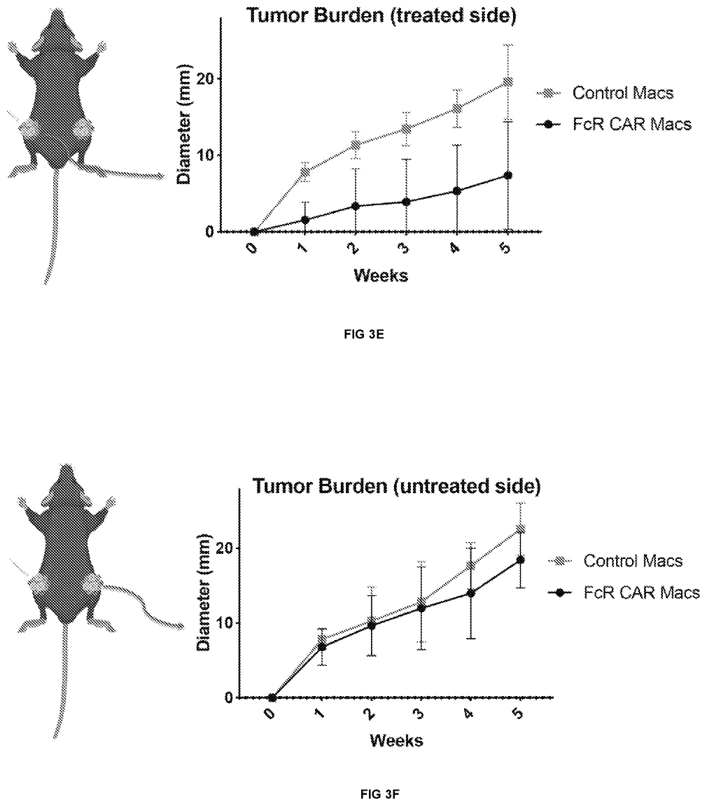

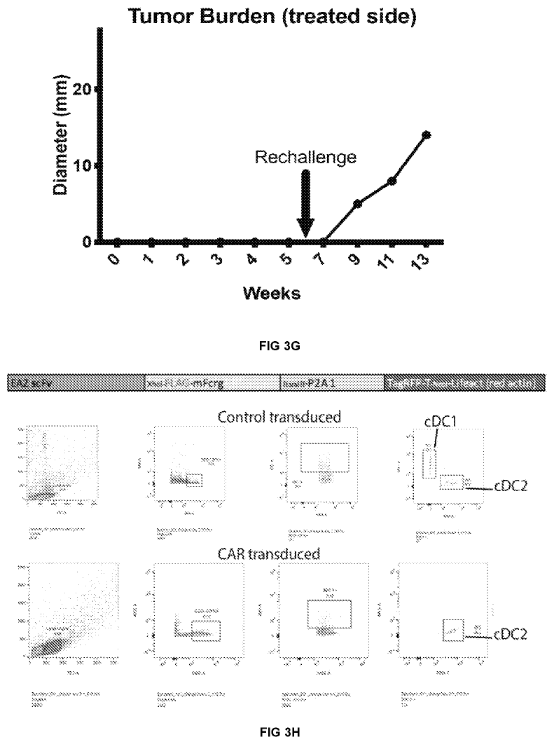

CAR T-cells are effective in treating people with blood cancers. However, these cells are not capable of effectively penetrating a solid tumor (see FIG. 1A). To develop alternates to CAR T-cells, use of antigen presenting cells like dendritic cells expressing CAR was explored (FIG. 1i). A chimeric antigen receptor against the EphA2 tumor antigen was used in these CARs for initial experimentation (FIG. 2A). The CAR construct was fused with RFP as a marker for expression. The CAR was optimized by changing the signal peptide, the extracellular domain, transmembrane domain and the intracellular signaling domains (FIG. 2B). The CAR constructs could be successfully transduced into bone marrow macrophages as indicated by the expression of RFP (see FIG. 3A). The CAR construct had an extracellular domain as in SEQ ID NO: 18, transmembrane domain as in SEQ ID NO: 19 and intracellular domain as in SEQ ID NO: 21 (ITAM based domain, corresponding human intracellular domain is provided in SEQ ID NO: 22).

In vivo studies using syngeneic single sarcoma and dual flank sarcoma mouse model showed that these CAR cells were effective in reducing the size of the tumor (see FIGS. 3B-3F). However, no effective T-cell memory was developed as evident from a rechallenge (FIG. 3G). This may be because traditional Fc receptor-based CARs introduced into myeloid precursor cells do not form conventional type 1 dendritic cells (cDC1s), but rather form macrophages, even when they are grown in the presence of the DC-differentiating cytokine Flt3L (FIG. 3G). One reason for this could be that the early FcR signaling negatively impacts cDC1 differentiation (FIG. 3H).

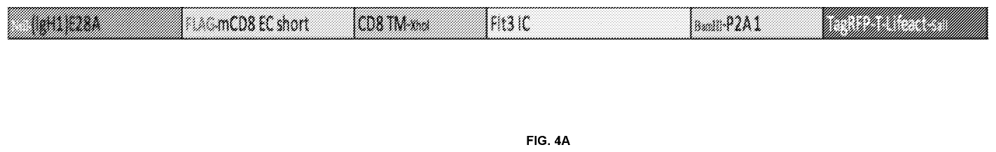

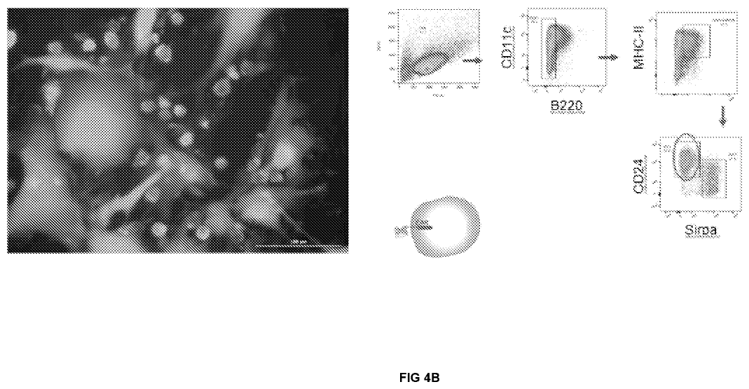

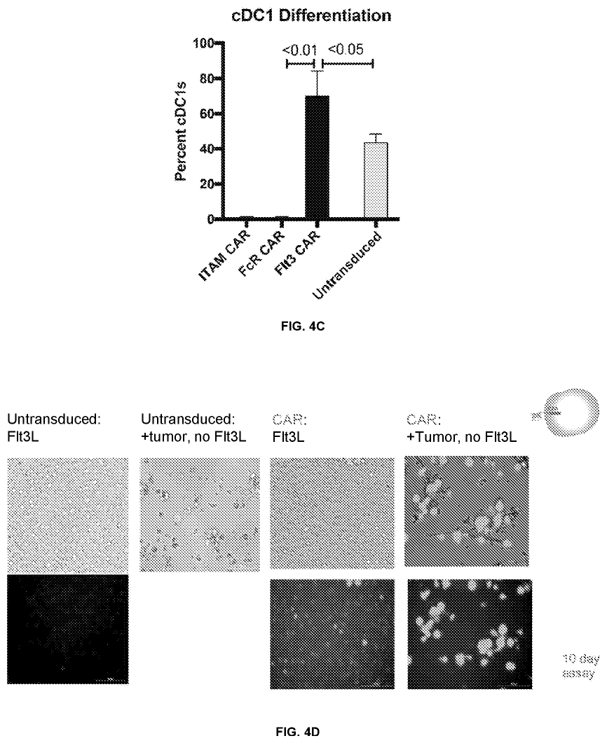

In order to develop effective CAR cDC1, the use of the intracellular domain of Flt3 receptor tyrosine kinase, that is important for cDC differentiation and survival in the bone marrow and periphery was tested. The construct used is provided in FIG. 4A. Fluorescence imaging and flow cytometric data show that the resulting dendritic cells successfully survived and differentiated in in vivo tumor microenvironment (FIGS. 4B-4D).

### Example 2: Effect of Extracellular Domain Length on Function of Car Dendritic Cells

For further optimization, the length of extracellular domains on the survival of CAR DCs on tumor and CAR-mediated tumor uptake, was tested.

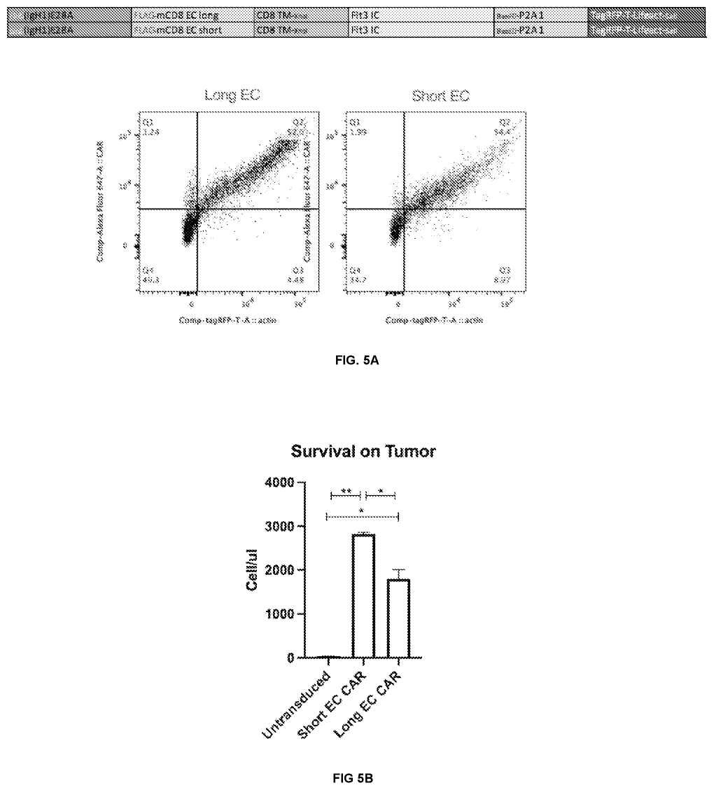

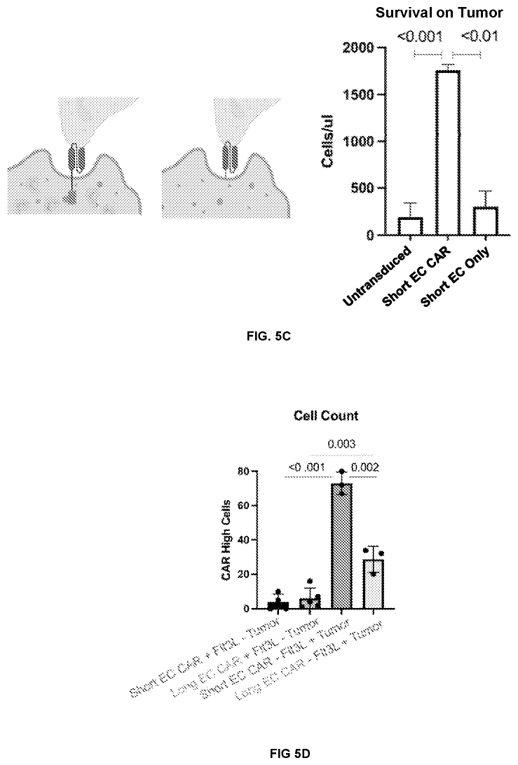

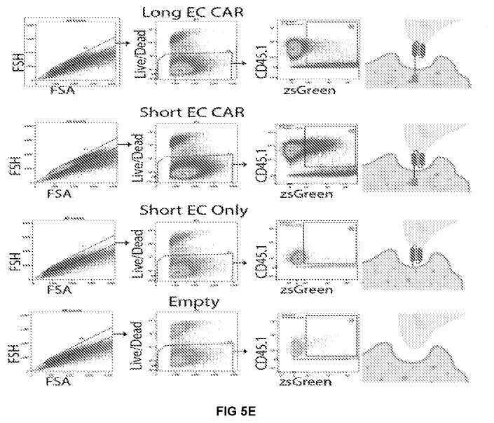

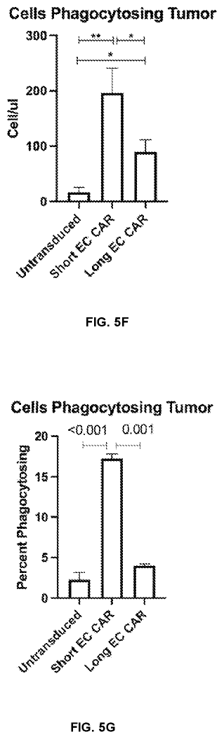

Using a CAR that targets the tumor associated antigen EphA2, Flt3 based CAR with either a short (13 amino acids total; 8 amino acids from a FLAG tag sequence DYKDDDDK, and 5 amino acids of the CD8 extracellular domain: SEQ ID. NO: 7) or long extracellular domain (43 amino acids total; 8 amino acids from a FLAG tag sequence DYKDDDDK and 35 amino acids of the CD8 extracellular domain, SEQ ID NO: 8) were generated. The remainder of the CARs expressed the CD8 transmembrane domain (SEQ ID NO: 11) followed by the Flt3 intracellular domain (SEQ ID NO: 5). The CAR constructs were introduced into a HoxB8 pluripotent DC precursor, which relies on Flt3L for survival. Both constructs resulted in similar CAR expression as seen in FIG. 5A. When grown on tumor cells that express the CAR target in the absence of Flt3L, nearly all un-transduced cells died as expected. However, the long DC CAR significantly improved survival of the DC precursors grown on tumor. Additionally, the short DC CAR performed substantially better in survival and phagocytosis assays (see FIG. 5B).

To test whether the beneficial effects of the short extracellular domain was related to its effect on intracellular signaling, or whether the interaction with tumor mediated by the short extracellular domain has beneficial effect in the absence of the intracellular domain, the short CAR was compared with an identical version that lacked the intracellular signaling domain in tumor coculture. The short EC CAR lacking signaling had no better survival than control untransduced cells, while short CAR with the Flt3 signaling domain again had substantially improved survival (FIG. 5C).

To further determine whether differences in CAR signaling occur upon tumor binding when using a Flt3 CAR with a short versus long extracellular domain, CAR transduced cells were co-cultured with tumor expressing the target antigen, in the presence or absence of exogenous Flt3L. The co-cultures were assessed for whether number of cells expressing a high level of CAR (quantified by fluorescence intensity) preferentially grew out over low CAR expressing cells after 10 days of coculture with tumor (FIG. 5D). In the absence of Flt3L high CAR expressing cells survived or proliferated to a much greater extent if they expressed a short extracellular domain rather than a longer one.

The Flt3 intracellular domain utilized here is not traditionally thought of as mediating phagocytosis, however in its receptor form it does exhibit receptor-mediated endocytosis when bound to its ligand. To test whether this DC CAR induces any tumor uptake upon binding, we co-cultured CAR HoxB8 DC cells with tumor expressing zsGreen for 48 hours. Surprisingly, the short CAR cDC1 promoted a substantial amount of tumor uptake, while the long CAR cDC1 resulted in less tumor uptake, and the non-signaling short CAR and untransduced cells both exhibited very limited tumor uptake (2-4%) (FIG. 5E-5F). This was also true both when percent of cells taking up tumor, or when the absolute number of cells taking up tumor (after plating equivalent numbers) were quantified (FIG. 5E-5G).

### Example 2: Effect of Promoter Driving Car Expression on DC1 Differentiation

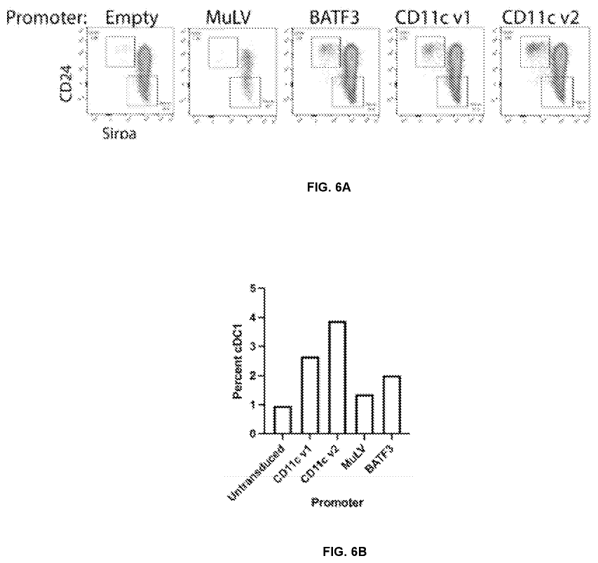

To assess the effects of the promotor sequence driving CAR expression in cDCs on DC1 differentiation, the following experiments were conducted. The MuLV promoter commonly used to drive expression of CAR in T-cells, is silenced in differentiated cDC1s. CD11c is a promoter active in cDCs. However, the elements of CD11c have not been tested in the context of promoting CAR expression in a cDC1. Various promoters were tested for the ability to continue to drive expression of CAR in differentiated cDC1s, using the MuLV promoter as baseline. These included MuLV with additional binding sites for transcription factors active in cDC1s (BATF3, IRF8, and NFIL3), a minimal CD11c based promoter (CD11c v1), and a minimal CD11c based promoter with additional BATF3 sites (CD11c v2) inserted upstream of the KOZAK and transcription start site. When transduced cells were fully differentiated to DCs in coculture with cells expressing the CAR target antigen and minimal exogenous Flt3L (1 ng/ml), CAR DCs being driven by the modified CD11c v2 promoter formed more DC1s than those driven by other promoters (see FIGS. 6A and 6B).

### Example 3: In Vivo Effect of Car DC1 Treatment in Syngeneic Sarcoma Mouse Model

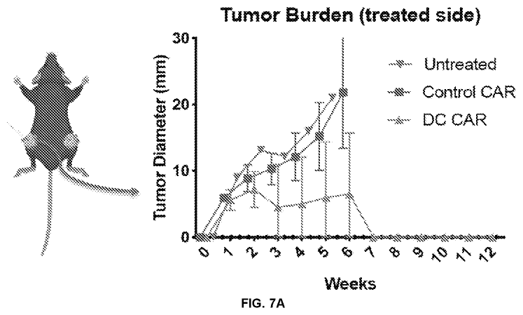

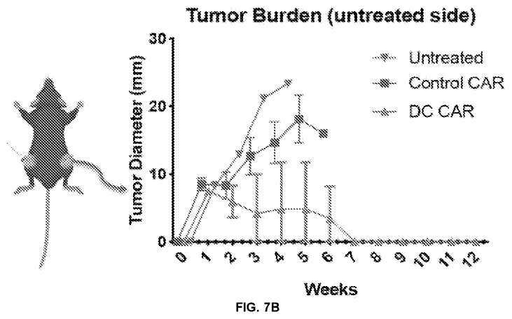

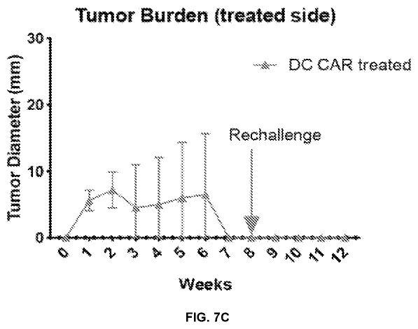

C57BL/6 mice were injected bilaterally in the flank with about 1×10⁶ mouse 1956 tumor cells, which naturally express EphA2. A single injection of 3×10⁶ EphA2 targeted CAR cDC1 (unsorted, in conjunction with 2.5×10⁶ cDC2s) with a short extracellular domain, or the same number of control cDC1s were injected into the tumor after 1 day. Tumor burden was measured as shown in FIGS. 7A and 7B for treated and untreated tumors respectively. All mice that exhibited a complete response were re-challenged with tumor, by injecting another 1×10⁶ tumor cells into the flank. Only mice in the EphA2 targeting short CAR DC group exhibited a complete response, and tumors were unable to subsequently grow in these mice upon re-challenge (FIG. 7C).

### Example 4: Effect of Extracellular Domain Length on Cross-Priming Ability of CD19-CAR cDC1

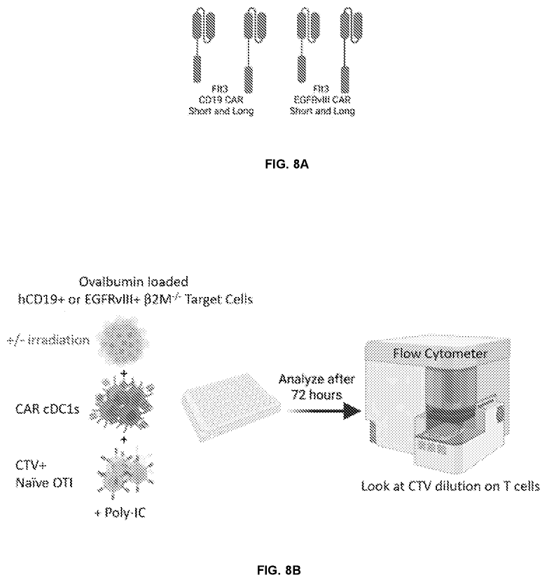

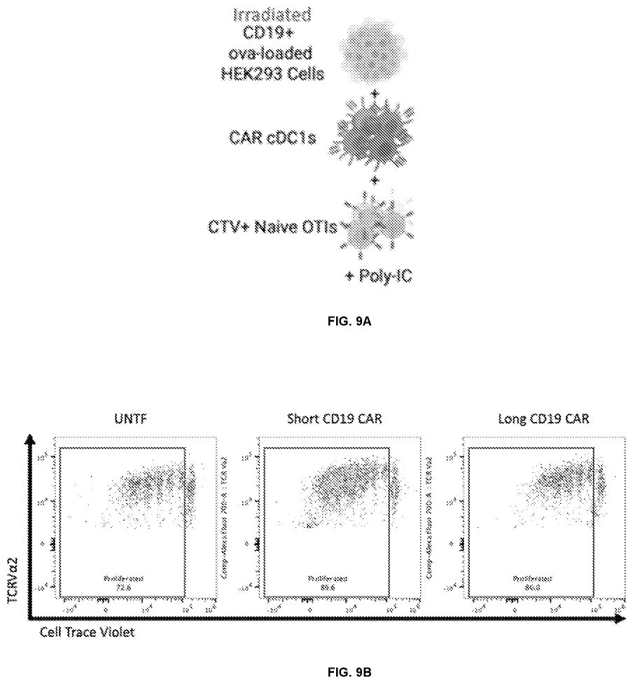

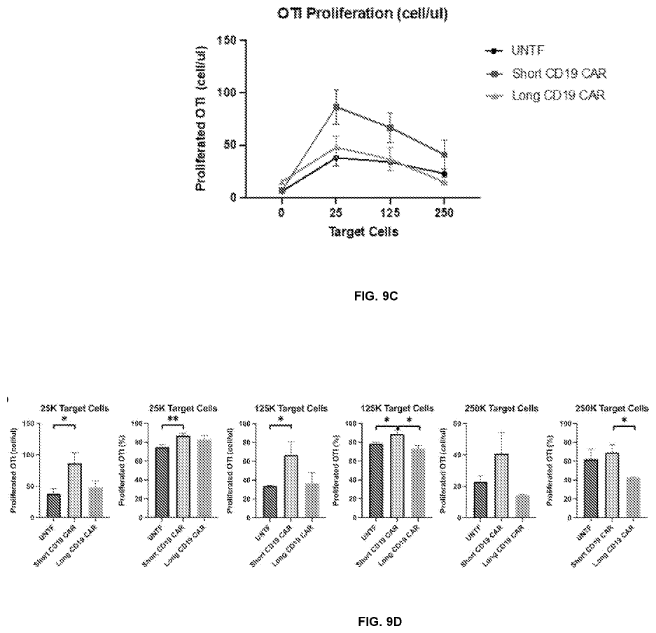

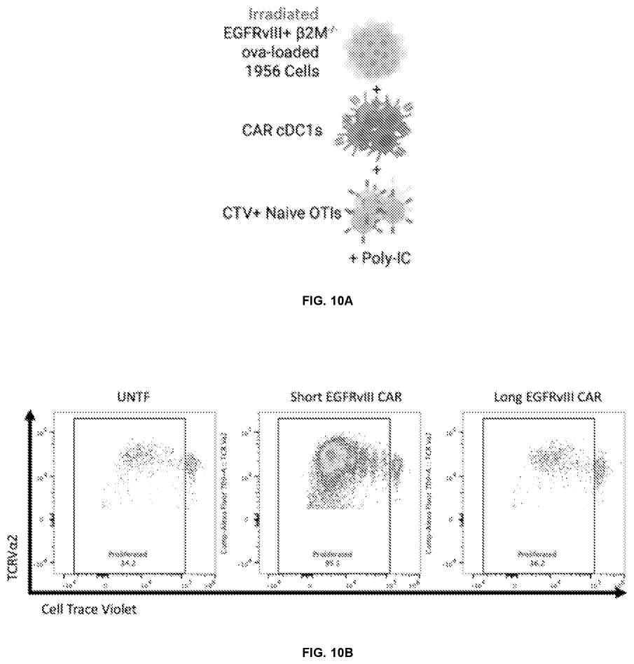

Additional CAR combining either CD19 or EGFRVIII targeting antibodies with long or short EC domains and the Flt3 signaling domain were constructed essentially as provided in the Methods (FIG. 8A). A cross priming assay were conducted by combining ovalbumin loaded target cells (OTI) that express either human CD19 or EGFRvIII and do not express MHC-I, with CAR DC, CTV-stained naïve OTI (TCR transgenic CD8 T cells that recognize MHCI:SIINFEKL) T cells, poly-IC (TLR3 agonist) (FIG. 8B). Flow cytometry experiments were conducted 72 hrs after coculture gated on CD45.1⁺mRuby⁻CD8a⁺TCRVa2+(FIG. 9A). FIG. 9B shows representative flow plots of proliferated OTI cells 72 hours after coculture with CD19-CAR cDC1 with either short or long extracellular domains. Untransformed DCs were used as control. FIGS. 9C and 9D shows the quantitation of the flow cytometry data. Results show that the short CD19-CAR cDC1 outperformed the long CD19-CAR cDC1 in these assays.

### Example 6: Effect of Extracellular Domain Length on Cross-Priming Ability of CD19-CAR cDC1

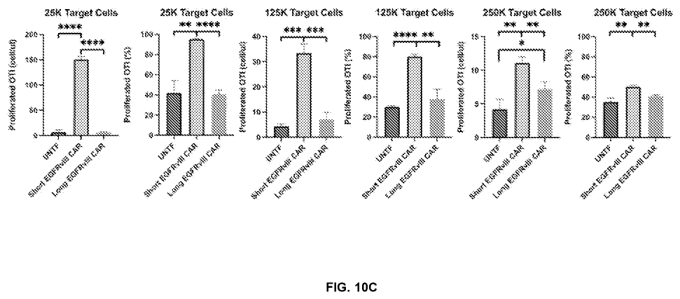

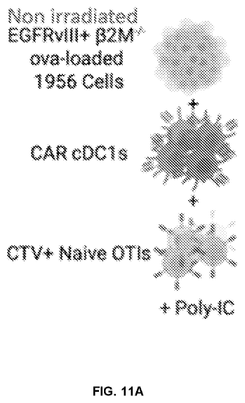

Similar experiments were conducted with EGFRvIII-CAR cDC1 with long or short extracellular domains. Dendritic cells were cocultured with irradiated (60Gy) ovalbumin loaded zsGreen⁺EGFRvIII⁺B2M⁻ mouse 1956 tumor cells and naïve CTV+OTI T cells with Poly-IC (FIG. 10A). Flow cytometry of proliferated OTI cells 72 hours after coculture gated on CD45.1⁺zsGreen⁻CD8a⁺TCRVa2 was conducted (FIG. 10B). Results show that as in the case with CD19-CAR DC, the EGFRvIII-CAR cDC1 with the short extracellular domain were more effective in inducing T-cell proliferation (FIG. 0C).

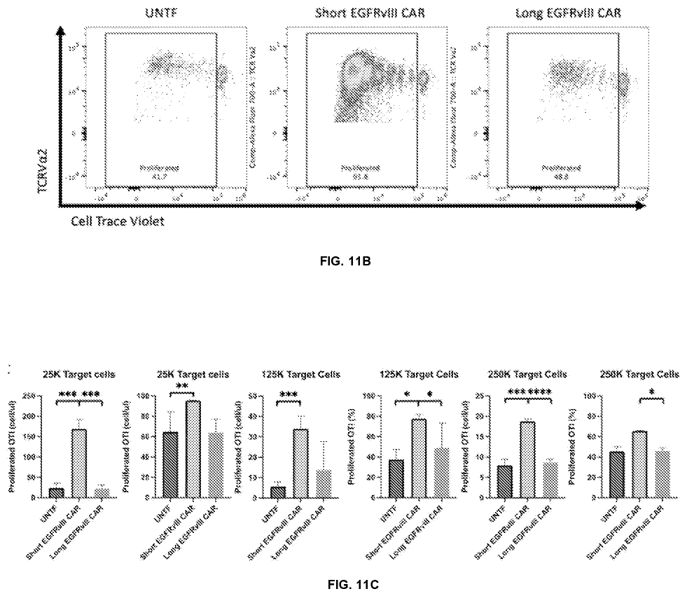

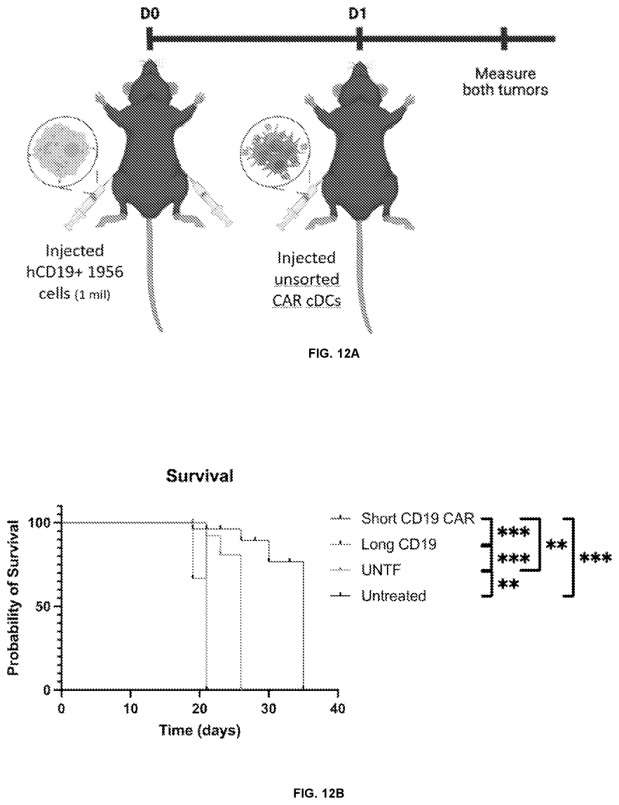

Next, cross-priming experiments were conducted using non-irradiated OTIs (FIG. 11A). Dendritic cells were cocultured with non-irradiated (60Gy) ovalbumin loaded zsGreen⁺EGFRvIII⁺B2M⁻ mouse 1956 tumor cells and naïve CTV+OTI T cells with Poly-IC (FIG. 11A). Flow cytometry of proliferated OTI cells 72 hours after coculture gated on CD45.1*zsGreen⁻CD8a⁺TCRVa2+ were conducted (FIG. 11B). As seen from the plots in FIG. 12C, the EGFRvIII-CAR DCs with the short extracellular domain were reliably successful in inducing OTI proliferation.

### Example 7: Effect of Extracellular Domain Length on Tumor Bearing Mouse Survival

Next, the in vivo mice experiments were conducted to test the relative difference in the impact of CD19-CAR cDC1 with long or short extracellular domains. C57BL/6 mice were injected bilaterally in the flank with about 1×10⁶ human CD19+ mouse 1956 tumor cells. About 7.5×10⁶ unsorted CD19-CAR cDC1 with short or long extracellular domains or control cDCs were injected into the tumor (FIG. 12A). Results showed significant increase in survival of mice injected with CD19-CAR cDCs with short extracellular domain in comparison to CD19-CAR cDCs with long extracellular domains (FIG. 12B).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1. A chimeric antigen receptor (CAR), comprising:
a) an intracellular signaling domain comprising an FMS-like tyrosine kinase 3 (Flt3) signaling domain or a functional fragment, variant, or derivative thereof; and
b) an extracellular domain no longer than 40 amino acids.

2. (canceled)

3. The CAR of claim 1, wherein the extracellular domain comprises a portion of a CD8 extracellular domain.

4.-5. (canceled)

6. The CAR of claim 3, wherein the portion of the CD8 extracellular domain comprises a polypeptide sequence at least about 80% identical to SEQ ID NO: 7, 8 or 18.

7. The CAR of claim 1, wherein the intracellular domain promotes survival and proliferation of dendritic cells.

8. The CAR of claim 7, wherein the intracellular signaling domain comprises a polypeptide sequence at least about 80% identical to SEQ ID NO: 5 or 6 or a functional fragment, variant, or derivative thereof.

9. (canceled)

10. The CAR of claim 1, further comprising an antigen binding domain.

11. (canceled)

12. The CAR of claim 10, wherein the antigen binding domain targets a tumor antigen.

13. The CAR of claim 12, wherein the tumor antigen is selected from a group consisting of BCMA, EphA2, B7H3, CD19 and EGFRvIII.

14. (canceled)

15. The CAR of claim 1, further comprising a transmembrane domain.

16. The CAR of claim 15, wherein the transmembrane domain is at least about 80% identical to any one of SEQ ID. NOS: 11, 12 13, 19 or 20.

17. The CAR of claim 15, wherein the CAR comprises an antigen binding domain, and wherein the extracellular domain links the antigen binding domain to the transmembrane domain.

18. A polynucleotide comprising a nucleic acid sequence encoding the CAR of claim 1.

19. The polynucleotide of claim 18, further comprising a promoter operably linked to the nucleic acid sequence encoding the CAR.

20. The polynucleotide of claim 19, wherein the promoter is a CD11c promoter or a variant or derivative thereof.

21. The polynucleotide of claim 19, wherein the promotor comprises a nucleic acid sequence at least about 60% identical to SEQ ID NO:1, SEQ ID NO:2, or SEQ ID NO:3.

22. A modified cell comprising the CAR of claim 1.

23. The modified cell of claim 22, wherein the modified cell is a dendritic cell.

24. The modified cell of claim 23, wherein the dendritic cell is selected from a conventional type 1 dendritic cell (cDC1) or a progenitor cell thereof.

25. (canceled)

26. The modified cell of claim 24, wherein the progenitor cell is selected from a peripheral blood mononuclear cell (PBMC), a CD34+ hematopoietic stem cell, a monocyte and dendritic cell progenitor (MDP), a common myeloid progenitor (CMP), a lymphoid-primed multipotent progenitor (LMPP), a common dendritic cell progenitor (CDP), or a stem cell.

27. The modified cell of claim 22, wherein the modified cell (a) cross presents a tumor antigen, elicits an adaptive antitumor immune response, or activates antitumor T cells: (b) the modified cell selectively engulfs tumor cells, cross-presenting a tumor antigen, or activating T-cells to respond to the tumor antigen; or (c) eliminates antigen positive (Ag+) tumors targeted by the CARs, and indirectly eliminating CAR-Ag− solid tumor cells (not recognized by the CAR), through epitope spreadinq.

28. (canceled)

29. (canceled)

30. A pharmaceutical composition comprising the CAR of claim 1, and a pharmaceutically acceptable excipient.

31. (canceled)

32. A method of treating cancer in a subject in need thereof, comprising administering to the subject the pharmaceutical composition of any-nee of claim 30.

33.-45. (canceled)

46. A chimeric antigen receptor (CAR), comprising from N-terminus to C-terminus:
a) a signal peptide;
b) an antigen binding domain;
c) an extracellular domain no longer than 40 amino acids;
d) a transmembrane domain; and
e) an intracellular signaling domain comprising a FMS-like tyrosine kinase 3 (Flt3) signaling domain or a functional fragment, variant or derivative thereof.

47.-58.

59. A CAR comprising an:
a) extracellular domain comprising an amino acid sequence no longer than 13 amino acids;
b) a tumor antigen binding domain, wherein the tumor antigen is selected from BCMA, EphA2, B7H3, CD19 and EGFRvIII;
c) a transmembrane domain comprising an amino acid sequence from a human CD8α or CD8b transmembrane domain; and
d) an intracellular domain comprising a FMS-like tyrosine kinase 3 (Flt3) signaling domain or a functional fragment, variant or derivative thereof.


SEQUENCE LISTING (22 sequences)

SEQ ID NO: 1 - DNA, 199 bp, synthetic construct
  misc_feature 1..199: note = Synthetic Sequence
  source 1..199: mol_type = other DNA
gtggaccatcctctagactggcggccgcatgacgtggcgctcgcgctttgaggatacattctgaacatgacgtggccgagcctggttgaggatagccgcgcagtccaatagctcagccccctgtggcggctccctcccggtcttcctatataagatgccctacgagcagcgccaccatggaatttggcctgagctgggt

SEQ ID NO: 2 - DNA, 173 bp, synthetic construct
  misc_feature 1..173: note = Synthetic Sequence
  source 1..173: mol_type = other DNA
tcggtccccactctatccatttcatctcagtttgcccccacctcctctgagtcacgctgacaacttccctcctggtctctggcctcctgaccacctttcttctcatttgcttcttctgtggtgacttggcagctgtctccaagttgctcagagcctgcttctgttctccagtc

SEQ ID NO: 3 - DNA, 258 bp, synthetic construct
  misc_feature 1..258: note = Synthetic Sequence
  source 1..258: mol_type = other DNA
gtggaccatcctctagactggcggccgctgaggatacgagcctggtatgacgtggctcggtccccactctatccatttcatctcagtttgcccccacctcctctgagtcacgctgacaacttccctcctggtctctggcctcctgaccacctttcttctcatttgcttcttctgtggtgacttggcagctgtctccaagttgctcagagcctgcttctgttctccagtcgccaccatggaatttggcctgagctgggt

SEQ ID NO: 4 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 428 aa, Mus musculus
HKYKKQFRYESQLQMIQVTGPLDNEYFYVDFRDYEYDLKWEFPRENLEFGKVLGSGAFGRVMNATAYGISKTGVSIQVAVKMLKEKADSCEKEALMSELKMMTHLGHHDNIVNLLGACTLSGPVYLIFEYCCYGDLLNYLRSKREKFHRTWTEIFKEHNFSFYPTFQAHSNSSMPGSREVQLHPPLDQLSGFNGNSIHSEDEIEYENQKRLAEEEEEDLNVLTFEDLLCFAYQVAKGMEFLEFKSCVHRDLAARNVLVTHGKVVKICDFGLARDILSDSSYVVRGNARLPVKWMAPESLFEGIYTIKSDVWSYGILLWEIFSLGVNPYPGIPVDANFYKLIQSGFKMEQPFYATEGIYFVMQSCWAFDSRKRPSFPNLTSFLGCQLAEAEEAMYQNMGGNVPEHPSIYQNRRPLSREAGSEPPSPQAQ

SEQ ID NO: 6 - protein, 430 aa, Homo sapiens
HKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 7 - protein, 5 aa, synthetic construct
CSLTT

SEQ ID NO: 8 - protein, 35 aa, synthetic construct
TTAPTKKTTLKMKKKKQCPFPHPETQKGLTCSLTT

SEQ ID NO: 9 - protein, 19 aa, Homo sapiens
MEFGLSWVFLVALFRGVQC

SEQ ID NO: 10 - protein, 21 aa, synthetic construct
MALPVTALLLPLALLLHAARP

SEQ ID NO: 11 - protein, 21 aa, Mus musculus
LSLLVVCILLLLAFLGVAVYF

SEQ ID NO: 12 - protein, 21 aa, Homo sapiens
IYIWAPLAGTCGVLLLSLVIT

SEQ ID NO: 13 - protein, 21 aa, Homo sapiens
ITLGLLVAGVLVLLVSLGVAI

SEQ ID NO: 14 - protein, 42 aa, Homo sapiens
PAPRPPTPAPTIASQPLSLRPEACRPAAGGAVHTRGLDFACD

SEQ ID NO: 15 - protein, 42 aa, Homo sapiens
GTQLSVVDFLPTTAQPTKKSTLKKRVCRLPRPETQKGPLCSP

SEQ ID NO: 16 - protein, 15 aa, synthetic construct
GGGGSGGGGSGGGGS

SEQ ID NO: 17 - protein, 8 aa, Mus musculus
SIINFEKL

SEQ ID NO: 18 - protein, 5 aa, synthetic construct
LGEPQ

SEQ ID NO: 19 - protein, 21 aa, Mus musculus
LCYILDAVLFLYGIVLTLLYC

SEQ ID NO: 20 - protein, 21 aa, Homo sapiens
LCYILDAILFLYGIVLTLLYC

SEQ ID NO: 21 - protein, 42 aa, Mus musculus
RLKIQVRKAAIASREKADAVYTGLNTRSQETYETLKHEKPPQ

SEQ ID NO: 22 - protein, 42 aa, Homo sapiens
RLKIQVRKAAITSYEKSDGVYTGLSTRNQETYETLKHEKPPQ